(19) United States
(12) Patent Application Publication
(10) Pub. No.: US 2023/0106738 A1
(43) Pub. Date: Apr. 6, 2023

(54) COMPOSITIONS, METHODS, AND KITS FOR DETECTING THE NUMBER AND GENOMIC LOCATIONS OF POLYMORPHIC LINE-1 ELEMENTS IN AN INDIVIDUAL

(71) Applicant: Administrators of the Tulane Educational Fund, New Orleans, LA (US)

(72) Inventors: Victoria PEREPELITSA, New Orleans, LA (US); Prescott DEININGER, New Orleans, LA (US)

(73) Assignee: Administrators of the Tulane Educational Fund, New Orleans, LA (US)

(21) Appl. No.: 17/802,523

(22) PCT Filed: Mar. 1, 2021
(86) PCT No.: PCT/US2021/020346

§ 371 Date: Aug. 25, 2022

Related U.S. Application Data
(60) Provisional application No. 62/982,596, filed on Feb. 27, 2020.

(51) Int. Cl. C12Q 1/6827; C12Q 1/6834; C12Q 1/6883; C12Q 1/6886

(52) U.S. Cl. CPC C12Q 1/6827; C12Q 1/6834; C12Q 1/6883; C12Q 1/6886

(57) ABSTRACT

The invention provides compositions, methods, kits, and devices for detecting the number and locations of polymorphic LINE-1 (pL1s) elements present in the genome of an individual and for detecting previously unknown pL1s. The inventive compositions, methods, kits, and devices permit the identification of numbers and patterns of pL1 insertions that render a person with such numbers and patterns at higher risk of developing cancer or cognitive disorders compared to persons without such numbers and patterns.

### CROSS-REFERENCE TO RELATED APPLICATIONS

This application claims priority to, and the benefit of, U.S. Provisional Patent Application No. 62/982,596, filed Feb. 27, 2020, the contents of which are incorporated herein by reference.

### STATEMENT OF FEDERAL FUNDING

This invention was made with government support under grant RO1 833AG057597 awarded by the National Institute on Aging of the National Institutes of Health. The government has certain rights in the invention.

### BACKGROUND OF THE INVENTION

Long Interspersed Element-1 (“L1”) retroelements are the only family of mobile genetic elements currently active in the human genome. See, e.g., Deininger et al., Nuc. Acids Res., 2017, 45(5):e31.doi:10.1093/nar/gkw1067 (hereafter, “Deininger”). About 500,000 L1 elements have accumulated in the genome over time and now comprise approximately 17% of human genomic content. See, e.g., Belancio et al., Nuc. Acids Res., 2010, 38(12):3909-3922; Lander et al., Nature, 2001, 409, 860-921, doi.org/10.1038/35057062. The majority of L1 elements in the genome are inactive, due either to truncation of their 5′ ends, mutations, or to internal rearrangements. There are, however, also a number of functional L1 elements which have both 5′- and 3′-untranslated regions (“UTRs”) and which do not contain inactivating rearrangements. Functional L1 elements continue to generate additional new copies in the genome of the individuals who carry them; the new L1 copies can then contribute to genetic instability during the individual's life, potentially increasing the individual's risk of diseases such as cancer or increasing the possibility that a cancer in the individual will be more aggressive than might otherwise be the case.

Many non-functional and some functional L1s are present in the genome of every individual. Since these L1s do not vary among individuals, they are sometimes referred to as “fixed L1s;” some fixed L1s are of a type identified as “PA2s,” or “L1PA2s.” As the fixed L1s do not vary in number between individuals, they are less likely to change the risk of genetic instability in any one individual compared to any other individual. “Polymorphic L1s,” on the other hand, vary in number from individual to individual. As polymorphic L1s, by definition, vary in number from one individual to another and they also vary in genomic position. Both the number and position of specific pL1s can place an individual at higher risk of genetic instability, and of diseases related to that genetic instability, than that of individuals with lower numbers of polymorphic L1s or with pL1s in other positions in their genome.

Unfortunately, there is currently no convenient, affordable method of screening patients to determine the number and genomic positions of polymorphic L1s they carry and to assess the individual's consequent risk of genetic instability. Surprisingly, the present invention fulfills these and other needs.

### BRIEF SUMMARY OF THE INVENTION

In a first group of embodiments, the invention provides compositions for determining how many polymorphic LINE-1 elements (“pL1s”) are present in genomic DNA of an individual subject, and at which sites within the individual's genome the pL1s are inserted. The pL1s have a 5′ untranslated region (“5′UTR”) and a 3′UTR, which 5′UTR begins with a contiguous sequence of at least 300 bases and which 3′UTR terminates in a contiguous sequence of at least 300 bases. In some embodiments, the composition comprises (a) a substrate or a plurality of substrates, (b) a plurality of first DNA probes, RNA probes, or both, attached to the substrate or the plurality of substrates, each of the DNA probes, RNA probes, or both, comprising a contiguous sequence of about 200 to about 1000 bases complementary to a consensus human genomic sequence surrounding and including one particular known pL1 insertion site, for each of the pL1 insertion points shown on Table 2, and (c) a plurality of second DNA probes, RNA probes, or both, which second DNA probes, RNA probes, or both, are complementary to the beginning contiguous sequence of the 300 bases of said 5′UTR of said pL1 or to said 3′UTR contiguous sequence of at least 300 bases. In some embodiments, the first DNA probes, RNA probes, or both, comprise a contiguous sequence of about 200 to about 700 bases. In some embodiments, the first DNA probes, RNA probes, or both, comprise a contiguous sequence of about 250 to about 500 bases. In some embodiments, the first DNA probes, RNA probes, or both comprise a contiguous sequence of about 300 to about 400 bases. In some embodiments, the substrate is a slide. In some embodiments, the substrate is a well of a multi-well plate. In some embodiments, the substrate is a wall of a microfluidic device. In some embodiments, some or all of the solid substrates are in the form of beads. In some embodiments, the solid surface is of quartz. In some embodiments, the solid surface is of glass. In some embodiments, the plurality of solid surfaces is of plastic. In some embodiments, the attachment of the first DNA probes or the second DNA probe, or both, to the solid support or the plurality of solid supports is covalent. In some embodiments, the composition further comprises (d) a plurality of third DNA probes, RNA probes, or both, attached to the substrate or the plurality of substrates, each of the third DNA probes, RNA probes, or both, comprising a contiguous sequence of about 200 to about 1000 bases complementary to a consensus human genomic sequence surrounding and including one or more particular fixed L1 insertion points associated with cancer.

In a further group of embodiments, the invention provides methods for determining how many polymorphic LINE-1 elements (“pL1s”) which pL1s have a 5′ untranslated region (“5′UTR”) and a 3′UTR, which 5′UTR begins with a contiguous sequence of at least 300 bases and which 3′UTR terminates in a contiguous sequence of at least 300 bases, may be full-length pL1s in genomic DNA of a subject who has both (a) pL1s, and (b) LINE-1 elements that occur at known genomic locations in all individuals (“fixed L1s”) with known genomic sequences upstream and downstream of said known genomic locations, and, with regard to the sites at which pL1s are known to insert in a human genome as shown in Table 2, at which of said sites at which said sites at which pL1s are known to occur pL1s are present in said subject, said method comprising the following steps, in the following order: (a) obtaining genomic DNA from said subject, which genomic DNA is fragmented into lengths of choice, and (b) contacting said fragmented genomic DNA with (1) a plurality of first DNA probes, first RNA probes, or a mixture of both first DNA probes and first RNA probes, each of which said first DNA probes and first RNA probes (i) comprises a contiguous sequence of about 200 to about 1000 bases complementary to a consensus human genomic sequence surrounding and including one particular known pL1 insertion site, wherein said plurality of said first DNA probes, first RNA probes, or mixture of both first DNA probes and first RNA probes taken together comprises human genomic sequence surrounding and including each of said pL1 insertion points shown in Table 2, and (ii) wherein each of said first DNA probes and said first RNA probes is (A) attached to an solid support or (B) are tagged with a tag which allows said probes to be specifically captured on a solid support when desired, and (2) a plurality of second DNA probes, second RNA probes, or mixture of both second DNA probes and second RNA probes, wherein said second DNA probes and said second RNA are complementary to said beginning contiguous sequence of said 300 bases of said 5′UTR of said pL1, further wherein each of said second DNA probe and second RNA probe is (A) attached to a solid support or (B) are tagged to allow said probes to be specifically captured on a support when desired, under conditions allowing said fragmented genomic DNA complementary to any of said first DNA probes, first RNA probes, or a mixture of both first DNA probes and first RNA probes or to said second DNA probes, second RNA probes, or a mixture of both second DNA probes and second RNA probes to hybridize to said probes, thereby creating a mixture of unhybridized fragmented genomic DNA, and fragmented genomic DNA that has hybridized to one of said probes, (c) if probes have been used in step (b) that are tagged to allow said tagged probes to be specifically captured on a solid support when desired, capturing said tagged probes on said solid support, (d) eluting any fragmented genomic DNA that has not hybridized to either one of said first DNA probes, first RNA probes, or mixture of both first DNA probes and first RNA probes, or one of said second DNA probes, second RNA probes, or mixture of both second DNA probes and second RNA probes, (e) eluting from said supports and collecting for sequencing any fragmented genomic DNA that hybridized to one of said first DNA probes, first RNA probes, or a mixture of both first DNA probes and first RNA probes, or to said second DNA probes, second RNA probes, or mixture of both second DNA probes and second RNA probes, thereby obtaining a plurality of previously-hybridized genomic DNA fragments,

(f) sequencing said plurality of previously-hybridized genomic DNA fragments, thereby obtaining a DNA sequence for each fragment contained within said plurality of previously-hybridized genomic DNA fragments, (g) comparing said DNA sequence for each fragment contained within plurality of previously-hybridized genomic DNA fragments to consensus human genomic sequences including each of said pL1 insertion sites set forth in Table 2, and determining for each of said pL1 insertion sites set forth in Table 2 whether:
(1) said genomic sequence upstream for each of said pL1 insertion sites is followed by (i) some or all of beginning of said L1 5′UTR sequence or (ii) some or all of said end of said L1 3′ sequence, indicating that for those insertion sites, there is a pL1 present that may be full length, and (2) whether said genomic sequence downstream for each of said pL1 insertion sites set forth in Table 2 is followed by (i) some or all of beginning of said L1 5′UTR sequence or (ii) some or all of said end of said L1 3′ UTR sequence, indicating that for those pL1 insertion sites, there is a pL1 present that may be full length. In some embodiments, the method comprises step (g)(3), compiling a list of how many pL1s that have said beginning of said L1 5′UTR and said end of said L1 3′UTR are present in said genome from said individual, thereby determining how many pL1s in said individual may be full-length. In some embodiments, the method further comprises step (g)(4), identifying in said list the locations of each of said pL1s. In some embodiments, the method further comprises step (g)(5), for each location in which a pL1 has been identified in step (g)(4), determining whether (A) said plurality of sequenced DNA sequences also contains a normal genomic sequence uninterrupted by a pL1 at said location, thereby determining that there is a copy of pL1 and a normal genomic sequence at that location, indicating that said genome of said individual has one copy of genomic sequence with said pL1 at said genomic location and one copy that does not have a pL1 at said location, or (B) said plurality of sequenced DNA sequences do not also contain a normal genomic sequence uninterrupted by a pL1 at said location, indicating that the genome of said individual has two copies of genomic sequence with said pL1 at said genomic location. In some embodiments, the method further comprises steps:
(h)(1), comparing the genomic sequences upstream and downstream of all L1 sequences in said plurality of sequenced DNA sequences to the genomic sequence upstream and downstream of said fixed L1s in said individual, (h)(2), determining how many fixed L1s have been detected compared to the number known to exist in the human genome, and (h)(3) reporting whether the number of fixed L1s detected in said individual is the same or different from the number of fixed L1s known to exist in said human genome. In some embodiments, the tag allowing the tagged probes to be specifically captured on a solid support is biotin or streptavidin. In some embodiments, the tag to allow said tagged probes to be specifically captured on a solid support is an antigen which is specifically bound by an antibody attached to said solid support. In some embodiments, the antigen is digoxigenin and the antibody is an anti-digoxigenin antibody.

In another group of embodiments, the invention provides methods for determining if an individual has a risk of developing cancer or Alzheimer's Disease due to polymorphic LINE-1 elements (“pL1s”) related to risk of cancer or Alzheimer's Disease in said individual's genome, said method comprising, determining if said individual carries one of more pL1s and, if so, how many, selected from the following groups: (a) pL1s identified in Table 2 as found by WGS, SCORE, or both, only in individuals diagnosed with breast cancer,

(b) pL1s identified in Table 2 as found by WGS, SCORE, or both, only in individuals diagnosed with prostate cancer,
(c) pL1s identified in Table 2 as found by WGS, SCORE, or both, in genomes of both individuals diagnosed with breast cancer and in genomes of individuals diagnosed with prostate cancer, but not in genomes of individuals listed in Table 2, column “Cont-WGS,”
(d) pL1s identified in Table 2 as found only in individuals diagnosed with Alzheimer's Disease,
(e) pL1s identified in Table 2 as found by WGS, SCORE, or both, in individuals diagnosed with Alzheimer's Disease, in individuals diagnosed with breast cancer, and in individuals diagnosed with prostate cancer, but not in genomes of individuals listed in Table 2, column “Cont-WGS,”
wherein, if said individual has one or more pL1s identified in groups (a)-(e), said individual is at risk of developing cancer or Alzheimer's Disease. In some embodiments, the pL1s are of group (a), and the individual's risk is of breast cancer. In some embodiments, the pL1s are of group (b), and the individual's risk is of prostate cancer. In some embodiments, the pL1s are of group (c), and the individual's risk is of cancer in general, (if female) breast cancer in particular, or, (if male) prostate cancer in particular. In some embodiments, the pL1s are of group (d), and the individual's risk is of Alzheimer's Disease. In some embodiments, the pL1s are of group (e), and the individual's risk is of cancer or Alzheimer's Disease.

In yet another group of embodiments, the invention provides methods for determining how many polymorphic LINE-1 elements (“pL1s”) which pL1s have a 5′ untranslated region (“5′UTR”) and a 3′UTR, which 5′UTR begins with a contiguous sequence of at least 300 bases and which 3′UTR terminates in a contiguous sequence of at least 300 bases, may be full-length pL1s in genomic DNA of a subject who has both (a) pL1s, and (b) LINE-1 elements that occur at known genomic locations in all individuals (“fixed L1s”) with known genomic sequences upstream and downstream of said known genomic locations, with regard to pL1 insertions sites at which pL1s are shown in Table 2 to be: (group 1) found to be inserted at said sites only in persons diagnosed with breast cancer, (group 2) found to be inserted at said sites only in persons diagnosed with prostate cancer, (group 3) found to be inserted at said sites in both persons diagnosed with breast cancer and in persons diagnosed with prostate cancer, (group 4) found to be inserted at said sites only in individuals diagnosed with Alzheimer's Disease, or, (group 5) found to be inserted at said sites in individuals diagnosed with Alzheimer's Disease, in individuals diagnosed with breast cancer, and in individuals diagnosed with prostate cancer, but not in genomes of individuals listed in Table 2, column “Cont-WGS”, said method comprising the following steps, in the following order: (a) obtaining genomic DNA from said subject, which genomic DNA is fragmented into lengths of choice, and (b) contacting said fragmented genomic DNA with (1) a plurality of first DNA probes, first RNA probes, or a mixture of both first DNA probes and first RNA probes, each of which said first DNA probes and first RNA probes (A) comprises a contiguous sequence of about 200 to about 1000 bases complementary to a consensus human genomic sequence surrounding and including one particular known pL1 insertion site, wherein said plurality of said first DNA probes, first RNA probes, or mixture of both first DNA probes and first RNA probes taken together comprises human genomic sequence surrounding and including each of said pL1 insertion points in at least one of said groups (1) to (5), and (ii) wherein each of said first DNA probes and said first RNA probes is (A) attached to an solid support or (B) are tagged with a tag which allows said probes to be specifically captured on a solid support when desired, and (2) a plurality of second DNA probes, second RNA probes, or mixture of both second DNA probes and second RNA probes, wherein said second DNA probes and said second RNA are complementary to said beginning contiguous sequence of said 300 bases of said 5′UTR of said pL1, further wherein each of said second DNA probe and second RNA probe is (A) attached to a solid support or (B) are tagged to allow said probes to be specifically captured on a support when desired, under conditions allowing said fragmented genomic DNA complementary to any of said first DNA probes, first RNA probes, or a mixture of both first DNA probes and first RNA probes or to said second DNA probes, second RNA probes, or a mixture of both second DNA probes and second RNA probes to hybridize to said probes, thereby creating a mixture of unhybridized fragmented genomic DNA, and fragmented genomic DNA that has hybridized to one of said probes, (c) if probes have been used in step (b) that are tagged to allow said tagged probes to be specifically captured on a solid support when desired, capturing said tagged probes on said solid support, or, if said probes were already attached to a solid support, proceeding to step (d), (d) eluting any fragmented genomic DNA that has not hybridized to either one of said first DNA probes, first RNA probes, or mixture of both first DNA probes and first RNA probes, or one of said second DNA probes, second RNA probes, or mixture of both second DNA probes and second RNA probes, (e) eluting from said supports and collecting for sequencing any fragmented genomic DNA that hybridized to one of said first DNA probes, first RNA probes, or a mixture of both first DNA probes and first RNA probes, or to said second DNA probes, second RNA probes, or mixture of both second DNA probes and second RNA probes, thereby obtaining a plurality of previously-hybridized genomic DNA fragments, (f) sequencing said plurality of previously-hybridized genomic DNA fragments, thereby obtaining a DNA sequence for each fragment contained within said plurality of previously-hybridized genomic DNA fragments, (g) comparing said DNA sequence for each fragment contained within plurality of previously-hybridized genomic DNA fragments to consensus human genomic sequences including each of said pL1 insertion sites for said in at least one of said groups (1) to (5), and determining for each of said pL1 insertion sites in said at least one of said groups (1) to (5) whether: (1) said genomic sequence upstream for each of said pL1 insertion sites is followed by (i) some or all of beginning of said L1 5′UTR sequence or (ii) some or all of said end of said L1 3′ sequence, indicating that for those insertion sites, there is a pL1 present that may be full length, and (2) whether said genomic sequence downstream for each of said pL1 insertion sites set forth in Table 2 is followed by (i) some or all of beginning of said L1 5′UTR sequence or (ii) some or all of said end of said L1 3′ UTR sequence, indicating that for those pL1 insertion sites, there is a pL1 present that may be full length. In some embodiments, the method further comprises step (g)(3), compiling a list of how many pL1s that have said beginning of said L1 5′UTR and said end of said L1 3′UTR are present in said genome from said individual, thereby determining how many pL1s in said at least one of said groups (1) to (5) may be full-length. In some embodiments, the methods further comprise step (g)(4), identifying in said list the locations of each of said pL1s in said at least one of said groups (1) to (5) present in said individual. In some embodiments, the methods further comprise step (g)(5), for each location in which a pL1 has been identified in step (g)(4), determining whether (A) said plurality of sequenced DNA sequences also contains a normal genomic sequence uninterrupted by a pL1 at said location, thereby determining that there is a copy of pL1 and a normal genomic sequence at that location, indicating that said genome of said individual has one copy of genomic sequence with said pL1 at said genomic location and one copy that does not have a pL1 at said location, or (B) said plurality of sequenced DNA sequences do not also contain a normal genomic sequence uninterrupted by a pL1 at said location, indicating that the genome of said individual has two copies of genomic sequence with said pL1 at said genomic location. In some embodiments, the methods further comprise steps: (h)(1), comparing the genomic sequences upstream and downstream of all L1 sequences in said plurality of sequenced DNA sequences to the genomic sequence upstream and downstream of said fixed L1s in said individual,

(h)(2), determining how many fixed L1s have been detected compared to the number known to exist in the human genome, and (h)(3) reporting whether the number of fixed L1s detected in said individual is the same or different from the number of fixed L1s known to exist in said human genome.

In still another group of embodiments, the invention provides electronic devices configured for determining how many polymorphic LINE-1 elements (“pL1s”) which pL1s have a 5′ untranslated region (“5′UTR”) and a 3′UTR, which 5′UTR begins with a contiguous sequence of at least 300 bases and which 3′UTR terminates in a contiguous sequence of at least 300 bases, are present in genomic DNA of a subject, and at which of the sites at which pL1s are known to insert said pL1s are present in said genomic DNA of said subject, said device comprising a processor and memory, said memory storing computer executable instructions for performing the methods of one or more the groups of embodiments set forth above.

In yet a further group of embodiments, the invention provides kits for determining, with regard to a human genome having a genomic sequence proceeding in direction from 5′ to 3′, which genome has known potential insertion points at which a full-length polymorphic LINE-1 element (“pL1”) may be inserted as set forth in Table 2, which of said insertion point has had a pL1 inserted, said full-length pL1s having a 5′ untranslated region (“5′UTR”) and a 3′UTR, which 5′UTR begins with a contiguous sequence of at least 300 bases and which 3′UTR terminates in a contiguous sequence, said kit comprising (a) a set of probes for all or substantially of said potential insertion points listed in Table 2, each member of which set of probes comprises (i) a sequence complementary to genomic sequence contiguous to one of said insertion points at which pL1 inserts into said genome, attached directly to a sequence complementary to at least the first 100 bases of said beginning of said 5′UTR of said pL1, and, (b) probes consisting essentially of 100-600 contiguous bases of said pL1 5′UTR.

In another group of embodiments, the invention provides kits for determining with regard to a human genome having a genomic sequence proceeding in direction from 5′ to 3′, which genome has 826 known potential insertion points at which a full-length polymorphic LINE-1 element (“pL1”) may be inserted which of said insertion point has had a pL1 inserted, said full-length pL1s having a 5′ untranslated region (“5′UTR”) and a 3′UTR, which 5′UTR begins with a contiguous sequence of at least 300 bases and which 3′UTR terminates in a contiguous sequence, said kit comprising (a) a set of probes for a subset of said 826 potential insertion points listed in Table 2, said subset consisting of one or more of said following groups:

group 1: pL1 insertions sites at which pL1s are shown in Table 2 to be found to be inserted at said sites only in persons diagnosed with breast cancer,
group 2: pL1 insertions sites at which pL1s are shown in Table 2 found to be inserted at said sites only in persons diagnosed with prostate cancer,
group 3: pL1 insertions sites at which pL1s are shown in Table 2 found to be inserted at said sites in both persons diagnosed with breast cancer and in persons diagnosed with prostate cancer,
group 4: pL1 insertions sites at which pL1s are shown in Table 2 found to be inserted at said sites only in individuals diagnosed with Alzheimer's Disease, and,
group 5: pL1 insertions sites at which pL1s are shown in Table 2 found to be inserted at said sites in individuals diagnosed with Alzheimer's Disease, in individuals diagnosed with breast cancer, and in individuals diagnosed with prostate cancer, but not in genomes of individuals listed in Table 2, column “Cont-WGS”, each member of which set of probes comprises (i) a sequence complementary to genomic sequence contiguous to one of said insertion points at which pL1 inserts into said genome, attached directly to a sequence complementary to at least the first 100 bases of said beginning of said 5′UTR of said pL1. In some embodiments, the kit further comprises (b) probes consisting essentially of 100-600 contiguous bases of said pL1 5′UTR. In some embodiments, the subset is the pL1 insertion sites of group 1. In some embodiments, the subset is the pL1 insertion sites of group 2. In some embodiments, the subset is the pL1 insertion sites of group 3.

### BRIEF DESCRIPTION OF THE DRAWINGS

FIGS. 1A-C. FIG. 1A. FIG. 1A is a schematic diagram illustrating aspects of designing probes for use in the inventive methods. The top thick line show a genomic sequence into which a first pL1, designated as pL1#1 in FIG. 1A, has inserted at a location annotated in the human genome. The original genomic sequence thus has been divided, with a section upstream of the pL1 and then continuing downstream of the inserted pL1. The thinner line immediately under the first thick line represents a probe designed to detect if a pL1 has inserted at this genomic location. The probe consists of has a first portion having the genomic sequence immediately upstream of the site at which the pL1 inserts, joined to the beginning of the pL1 5′UTR. The second thick line depicts the insertion of a pL1 at a second genomic location annotated in the human genome, and the design of a similar probe based on this second genomic location at which a pL1 inserts. The third thick line represents a design for a portion of genomic sequence at which a pL1 has been found to insert, but which has not yet been annotated in the consensus human genome as having a pL1. This design accommodates any pL1 which orientation in the genome is not known, as a pL1 may insert into the genome in either orientation. FIG. 1B. FIG. 1B shows the design of a probe to detect the presence of pL1s at sites at which pL1s are not known to insert, and to detect the presence of PA2s. FIG. 1C. FIG. 1C is a schematic diagram showing the overall flow of some embodiments of the inventive methods. The top diagram shows genomic DNA containing pL1s (shaded boxes) and a fixed L1s of the type referred to as “PA2” (horizontally striped box) present in the genome. After the DNA is fragmented (in this hypothetical example, by being sheared), it is hybridized to the probes of the various types described for FIGS. 1A and 1B. The DNA that hybridized to a probe is then amplified, sequenced, and analyzed by bioinformatics to determine the sites at which pL1s and PA2s have been detected as present in the genome.

FIG. 2. FIG. 2 is a graph showing the detection of PA2 fixed L1 elements in genomes in publicly available data sets analyzed by others by whole genome sequencing (“WGS”), or analyzed in the studies reported below using the inventive methods (“SCORE,” an acronym for the phrase “Screen for Content Of Retro Elements”). Controls: persons who have not been diagnosed with cancer or Alzheimer's Disease prior to genome analysis. Alzheimer's Disease: genomes from individuals diagnosed with Alzheimer's Disease prior to genome analysis. Breast cancer: genomes from individuals diagnosed with breast carcinoma prior to genome analysis. Prostate cancer: genomes from individuals diagnosed with prostate adenocarcinoma prior to genome analysis. Each circle represents a single genome.

FIG. 3. FIG. 3 is a graph showing the detection of pL1s in the same genomes as described for FIG. 2. FIG. 3 shows that the inventive methods (“SCORE”) detect considerably more pL1s than are detected by conventional analysis by whole genome sequencing (“WGS”).

FIG. 4. FIG. 4 is a graph showing pL1s that are present in the same genomes as described for FIG. 2, after subtracting pL1s at locations at which a pL1 was present in at least one genome in each group. Use of the inventive methods (“SCORE”) results in detecting numerous pL1s that were not detected by conventional whole genome sequencing (“WGS”). Data are presented as mean with standard deviation bars. Data were analyzed by two-tailed Student's t-test for n=2 groups. Significant P-values are indicated as follows: **** P<0.0001,*** P<0.005, ** P<0.01, * P<0.05. Statistical analysis was performed with GraphPad Prism.

FIG. 5. FIG. 5 is a graphical depiction of data regarding pL1s presented in Table 2. Th graph shows the number of pL1s detected in genomes in persons who had been diagnosed with Alzheimer's Disease, Breast Cancer, or Prostate Cancer, or who had not been diagnosed with one of these conditions (“Control”), after excluding pL1s present in at least one genome in each group. WGS and SCORE results were combined for breast cancer and for prostate cancer.

### DETAILED DESCRIPTION

### Introduction

As discussed in the Background, there are some 500,000 Long Interspersed Element-1 (“L1”) retroelements in the human genome. Of these approximately 500,000 L1 elements, only some 5000 are full length elements that contain the internal promoter, see, Deininger, supra, Lander et al., supra, and fewer have been identified as being active; that is, they have both 5′- and 3′- UTRs and no inactivating rearrangements and are capable of introducing new copies of themselves into the genome. Many functional L1 elements have inserted themselves at known positions in the human genome and are present in all human genomes in the same number. Functional and non-functional L1 elements present in all human genomes in the same number are sometimes referred to herein as “fixed L1s,” “PA2s,” or “L1PA2s” (fixed L1s and PA2s will be discussed in more detail in a later section). In addition to the fixed L1 elements, however, there are also full-length L1s retroelements that vary in number between individuals. Any one individual may have a different number of these L1 elements compared to others. Moreover, any one individual may have a different subset of locations at which these active L1 elements have inserted in their genome compared even to another individual with the same overall number of such L1s. The active L1 elements that vary in number and location among individuals are sometimes called “polymorphic” or “hot” L1 elements, as they can generate new integration events. See, e.g., Deininger, supra. L1s elements that vary in number among individuals will sometimes be referred to herein as “polymorphic L1s” or as “pL1s.”

By definition, functional L1 elements can continue to insert additional copies into the genome of individuals who carry them, and can contribute to genetic instability during the individual's life. Such new insertions potentially increase the individual's risk of developing diseases such as cancer. Further, pL1s insertions in a portion of the genome specifically expressed in a particular tissue or organ can increase the possibility that a cancer in that tissue or organ will be more aggressive than might otherwise be the case, and require more aggressive or different treatment than might otherwise be the case.

As the number of fixed L1s is the same among all individuals, the risk of genetic instability posed by fixed L1s is likely to similar for all individuals. But the number and specific locations of polymorphic L1s by definition varies from individual to individual, and a higher number of pL1s places carriers at a higher risk of genetic instability compared to those with lower numbers. For example, a person with a small number of pL1s would be considered at lower risk of genetic instability due to L1-associated mutations, while someone with a higher number of pL1s would be considered at higher risk of genetic instability. Further, persons with a low number of pL1s in their genomes, but with inherited defects in DNA repair pathways (especially those already known to increase the risk of developing cancer), would be considered at a higher risk for genetic instability from L1 than those without such genetic defects, because various DNA repair pathways guard against L1-induced genomic alterations. Moreover, the particular locations at which a pL1 has inserted is also important. As discussed further below, we have discovered that some pL1s inserted at some genomic locations are more likely to be associated with cancers or with Alzheimer's Disease than others, and that persons with pL1s inserted at the particular subsets of sites identified below therefore should be monitored more closely for development of cancer or Alzheimer's Disease than persons without pL1 insertions at these locations in the genome.

Persons with higher risk of genetic instability from pL1s might therefore benefit from being having more frequent medical checkups, starting from a younger age. Those with a high number of pL1s should also be considered in a number of pathologies other than cancer or Alzheimer's Disease. Among these are neurodegenerative diseases, infertility with unknown etiology, spontaneous abortions with unknown etiology, and sporadic genetic diseases with unknown etiology. Similarly, a person newly diagnosed with cancer who is determined to have a low number of pL1s in their genome might respond better to treatment than a person newly diagnosed with cancer who has a high number of pL1s in their genome.

Unfortunately, there is currently no convenient, affordable, and reproducible means for identifying the number of pL1s present in a particular individual, or for identifying patients with pL1 elements in a particular tissue type. Currently, the most direct method is to sequence the individual's entire genome in a procedure called whole genome sequencing, and to use bioinformatics programs to, first, search for each copy of full-length L1 and, second, determine which are fixed and which are in genomic positions at which the presence of an L1 retrotransposon is variable. While the price of whole genome sequencing has been dropping rapidly, sequencing the 3 billion +bases of the entire human genome to an informative depth is too expensive and time consuming for wide scale or routine screening, and render whole genome sequencing unsuitable for high throughput screening.

Surprisingly, the present invention solves these problems. In various embodiments, the invention provides methods and devices for determining which of the genomic locations at which pL1 elements that have found to insert to date are occupied by a pL1 in a subject, without the need for whole genome sequencing. Further, the methods, devices, and kits can not only identify which genomic locations at which pL1 elements are known to insert are in fact occupied by a pL1 element in a given subject, but also can determine whether the subject is heterozygous or homozygous with respect to a particular genomic location (that is, for the diploid chromosomes, whether a pL1 has inserted at a particular genomic location on both of the copies of the individual's chromosome, or just one). Moreover, the methods, devices and kits allow determining if the individual has any pL1s present that have not been previously identified. And, the methods, devices and kits include internal controls that allow the practitioner to determine if the assay is valid or whether the information provided is suspect due to, for example, a problem with the reagents or with storage of the DNA used as a patient sample. And, because the methods, devices and kits do not require whole genome sequencing, they are not only cheaper and faster than whole genome sequencing-based techniques, but they are also more sensitive and can also be used for high-throughput screening. In sum, the inventive methods, devices, systems, and kits provide a surprising combination of advantages that have not previously been available in the art.

One problem with whole genome sequencing, or “WGS,” is that cost considerations usually constrain the number of cycles that the practitioner has run on a genomic sample. This depth of sequencing is set by the practitioner at the beginning and is often not sufficient to detect all pL1s present in a sample, particularly those present in low allelic frequency. The studies reported below show that the inventive techniques uncovered pL1s at sites at which they were not located by WGS performed on other individuals with the same general diagnosis.

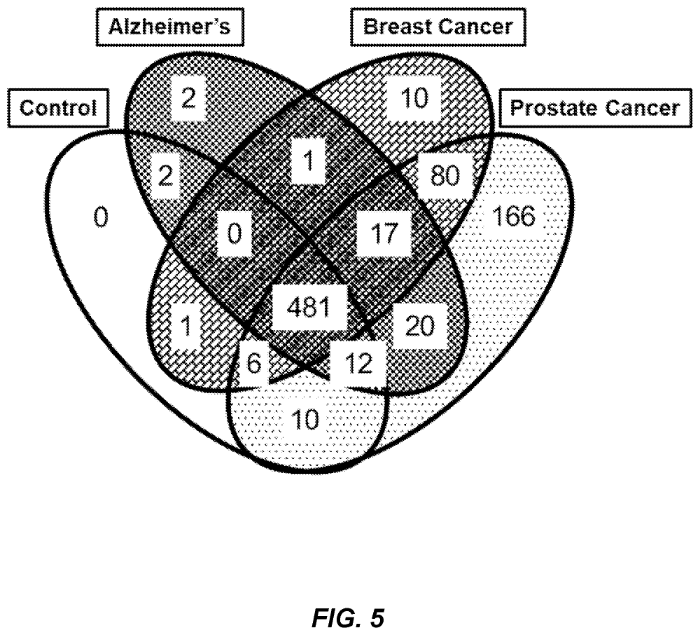

Further, our studies show that genomes of persons with breast cancer and prostate cancer had more pL1s present than did available data regarding the genomes of persons diagnosed with Alzheimer's Disease (“AD”) or who had not been diagnosed with either AD or with breast or prostate cancer (persons in this latter group will sometimes be referred to below as “controls”). Further, we found that persons with breast cancer or with prostate cancer had pL1s present in genomic locations at which pL1s had not inserted themselves in persons with AD or in controls. FIG. 5 presents the results of our analysis of pL1s present in genomes from persons with AD, breast cancer, prostate cancer, or controls. The Figure shows that genomes from persons with prostate cancer had pL1s present at 166 locations that were not shared with controls or with persons in the other groups studied, while genomes from persons diagnosed with breast cancer had 10 pL1s at positions in which they were not found in any of the genomes from persons in the other groups, and 80 pL1s at positions that were also locations of pL1s in genomes from persons who had been diagnosed with prostate cancer. Moreover, while the genomes from persons diagnosed with AD had pL1s at only 2 locations unique to that disorder in these datasets, the genomes had 1 pL1 at a location at which a pL1 was also found in persons with breast cancer, 17 with persons who had either breast cancer and prostate cancer, and 20 more with persons who had prostate cancer that were not also found in genomes from individuals diagnosed with breast cancer. Further, genomes from persons with prostate or breast cancer had more polymorphic L1s than did persons with AD or controls. And, reviewing the ages of the individuals, it was noted that persons 70-80 years old had fewer pL1s than did persons 40-70 years old who had been diagnosed with breast cancer or prostate cancer. On the other hand, no difference in the number of pL1s was noted between controls and persons with late-onset AD.

The results of the studies reported here show that the inventive methods make possible determining the number and distribution of pL1s in the genome of individuals, and that differences in the number and distribution of pL1s in the individual's genome can be used to determine whether they are more or less likely to develop cancer. In particular, as shown in FIG. 5, the finding of pL1s at certain genomic locations was found to be correlated with breast cancer or with prostate cancer, respectively. Thus, individual's whose genomes have pL1s at some or all of the genomic locations noted should be monitored for cancer in general and, depending on the sex of the individual and the particular locations at which the pL1s are present, for breast cancer or prostate cancer, respectively. The full list of genomic locations at which pL1s are known to insert into the genome is set forth in Table 2, as are the particular genomic locations at to which the presence of a pL1 was associated with breast cancer, prostate cancer, or AD.

### L1, the Human Genome, and pL1 Insertion Sites

As noted, active L1s have an internal promoter, both a 5-UTR and a 3′-UTR, and no inactivating rearrangements; they are therefore capable of introducing new copies of themselves into the genome. The full-length sequence of L1 is known in the art and available in references such as Scott et al., Genomics. 1987; 1(2):113-25 and Boissinot et al., Molecular Biol and Evolution, 2000; 17(6):915-928. Studies of the evolution of L1 elements in the human genome have resulted in further categorization of the elements as belonging in the PA subfamilies or in the subfamily HS. L1PA elements are fixed, while L1HS elements are considered to be younger, with some being fixed and some being polymorphic. Thus, the majority of fixed L1 elements are members of the PA subfamily, while all polymorphic 1s are in the HS subfamily. Fixed L1s are considered to be older in terms of the length of time they have been present in the human genome, and therefore have had more time in which to develop mutations. Some of these mutations can result in frame shifts or other changes that render the L1 incapable of introducing new copies of themselves. Fixed L1s with such mutations are, by definition, inactive. The sequence of any particular fixed or polymorphic L1 can be readily reviewed to determine if it is active or if mutations have rendered it inactive.

The sequence of the human genome was first published by Lander et al., Nature, 2001, 409:860-921 (references herein to the “human genome” or to the “genome” refer to the nuclear genome, not to the mitochondrial genome). The Genome Reference Consortium (“GRC”) currently maintains on GenBank a curated, publicly accessible, consensus reference genome sequence. As of this writing, the consensus sequence is Human Build 38 patch release 13 (GRCh38.p13), GenBank assembly accession: GCA_000001405.28; RefSeq assembly accession: GCF_000001405.39. The reference genomic sequence for each chromosome is available on GenBank, as set forth in Table 1.

TABLE 1

| Chromosome name | GenBank sequence | RefSeq sequence |
|---|---|---|
| 1 | CM000663.2 | NC_000001.11 |
| 2 | CM000664.2 | NC_000002.12 |
| 3 | CM000665.2 | NC_000003.12 |
| 4 | CM000666.2 | NC_000004.12 |
| 5 | CM000667.2 | NC_000005.10 |
| 6 | CM000668.2 | NC_000006.12 |
| 7 | CM000669.2 | NC_000007.14 |
| 8 | CM000670.2 | NC_000008.11 |
| 9 | CM000671.2 | NC_000009.12 |
| 10 | CM000672.2 | NC_000010.11 |
| 11 | CM000673.2 | NC_000011.10 |
| 12 | CM000674.2 | NC_000012.12 |
| 13 | CM000675.2 | NC_000013.11 |
| 14 | CM000676.2 | NC_000014.9 |
| 15 | CM000677.2 | NC_000015.10 |
| 16 | CM000678.2 | NC_000016.10 |
| 17 | CM000679.2 | NC_000017.11 |
| 18 | CM000680.2 | NC_000018.10 |
| 19 | CM000681.2 | NC_000019.10 |
| 20 | CM000682.2 | NC_000020.11 |
| 21 | CM000683.2 | NC_000021.9 |
| 22 | CM000684.2 | NC_000022.11 |
| X | CM000685.2 | NC_000023.11 |
| Y | CM000686.2 | NC_000024.10 |

While the genome of individuals differs from that of the reference genome, for example, by the presence of single nucleotide polymorphisms and the genetic variation that causes differences between individuals, that variation is not expected to significantly affect the conduct or performance of the inventive methods or devices.

As of this writing, over 800 sites in the human genome have been identified as positions at which a pL1 has been found. The sites can be referred to by their positions in the respective chromosomes and can conveniently be identified by reference to the genome sequence set forth in GenBank. Table 2, below, identifies the insertion points with respect to HumanBuild assembly 19 of the over 800 known pL1 insertion points, as well as a few fixed L1 locations (some of which are designated by being preceded by asterisks) reported to be active in certain cancers. As noted above, the current build is 38, patch release 13.

As the current build of the human genome in GenBank changes over time, persons of skill are accustomed to translating positions in any previous assembly of the genome to the current assembly, and various tools have been created to facilitate translating information from previous assemblies to more current ones. For example, the University of California, Santa Cruz (“UCSC”) maintains an on-line tool suite which it calls the Genome Browser. See, e.g, Kent et al., “The human genome browser at UCSC,” Genome Res. 2002, 12(6):996-1006; Karolchik et al., “The UCSC Table Browser data retrieval tool,” Nucleic Acids Res. 2004, 1;32(Database issue):D493-6. In particular, the UCSC Lift Genome Annotations tool (https://geonome.ucsc.edu/cgi-bin/hgLiftOver) converts genome coordinates and genome annotation files between assemblies and can be used to convert the coordinates set forth in Table 2 (assembly 19) to newer assemblies as they are developed.

As persons of skill appreciate, each human nucleated somatic cell carries two copies of each chromosome. Further, even if a pL1 is present in an individual's genome, to be capable of replication, it must be full length, which means it must have a full L1 5′ UTR.

The genome of an individual can exist in one of several states (with respect to pL1) with respect to each site on each chromosome at which polymorphic L1s have been found to date. First, the genome may be the normal sequence of the chromosome on both copies of the chromosome carried by that individual at the particular site a pL1 can insert: in that case, a pL1 is not present at that site in either copy of that individual's chromosome. Second, one copy of the chromosome at that site can have the normal genomic sequence, but the other copy of the chromosome can a genomic sequence interrupted by the presence of the sequence of a pL1, which shows that one copy of the individual's chromosome carries a pL1 at that location. Third, the normal sequence of both copies of the chromosome at that site may be interrupted by the presence of the sequence of a pL1, in which case the sequence shows that a pL1 is present in both copies. The fact that a pL1 sequence is present in one or both copies of the chromosome at that site does not necessarily mean it is active. If the pL1 sequence at the site does not commence with the start of the L1 5′ UTR, it is not a full-length sequence, and cannot be active.

As noted above, as of this writing, there are over 800 sites in the human genome at which pL1s have been reported to have been found to be inserted. Analyzing the sequence of an individual's genome on either side of a point in the genome at which a pL1 is known to insert therefore allows the practitioner to determine whether or not a pL1 is present in that individual at that genomic location on at least one of the individual's two copies of the chromosome on which that genomic location occurs. In some embodiments, the inventive methods and devices allow that determination to be made conveniently for each of the over 800 pL1s that have been identified to date, to identify if the individual has a pL1 copy on one copy of a given chromosome or a pL1 present on both copies of the chromosome, and to identify if the individual bears any pL1s that have not been identified to date. Additional pL1 insertions are still being found from time to time as research into the genome and LINE-1 elements continues. It is anticipated that any such new pL1 insertion points will be added to the list of known pL1 insertion points so that it can be also be determined whether a subject has a pL1 at the newly-known insertion point on one or both copies of the chromosome on which the new pL1 insertion point has been identified. It is further anticipated that in embodiments of the inventive methods using a probe set to detect the full set of sites at which pL1s have inserted into an individual's genome, probes for such newly identified sites will be added to the probe set to improve the diagnostic power of the methods and of devices using them to analyze the resulting genomic information.

This section will first present a brief overall of some embodiments of the inventive methods, followed by a more detailed discussion of some aspects. The entire human genome was first sequenced in 2001, and almost all of the sequences of all 22 numbered chromosomes and of the X and the Y chromosomes have been identified. As noted above, the over 800 currently-identified sites at which pL1s have been found to insert into the genome are also known, as is the normal genomic sequence at each site if a pL1 has not inserted itself at that site. For convenience of reference, the position in the sequence of a chromosome at which a particular pL1 element inserts into the normal genomic sequence is sometimes interchangeably referred to herein as the “pL1 insertion point” or the “pL1 insertion site.” The portions of the genomic sequence adjacent to the presence of a pL1 can be referred to as being upstream (“5″”) or downstream (“3′”) of the insertion point, respectively. For clarity with respect to later portions of the discussion, it is noted that a pL1 may be inserted into any particular insertion site in a subject's genome in either orientation (that is, 5′ to 3′ or 3′ to 5′). Thus, the 5′ portion of the subject's genomic sequence adjacent to the inserted pL1 may be adjacent to the 3′ end of the pL1, while the 3′ portion of the genomic sequence adjacent to the inserted pL1 may be adjacent to the pL1′s 5′UTR, or vice versa. Further, as noted elsewhere herein, while the sequence of all pL1s is the same or closely the same as all others, there are over 800 locations in the genome at which a pL1 has been found to insert. For clarity, references to an individual having a given number of pL1s refer not to different types of, or variations between, the pL1s, but to the number of locations in that individual's genome at which a pL1 was found to have been inserted.

In some embodiments of the inventive methods, a sample containing genomic DNA from the subject is obtained (references to a “subject,” “individual” or “patient” herein refers to a human subject). The sample can be obtained from any part of the subject's body, and can be taken prenatally (in the case of genetic testing of a fetal genome), shortly after birth, or at any time thereafter during the subject's life.

Collection of DNA from an individual is routine. Fetal DNA can be collected by, for example, amniocentesis. Post-natally, it is conveniently performed by swabbing the inside of the subject's cheek with a cotton swab, collection of a blood sample, or by taking a biopsy of any part of the individual (including, for neonates or for persons whose umbilical cord has been preserved, their umbilical cord). The genomic DNA is then isolated and fragmented, typically by shearing. The technique is generally selected and performed so as to result in randomly-generated segments of genomic DNA (for convenience of reference, hereafter referred to “sheared DNA”) which have been sheared to a length falling within maximum and minimum limits chosen by the practitioner. Typically, the practitioner will choose a maximum length that is convenient and cost effective to sequence using the techniques available at the time the DNA fragments are being sequenced, and a minimum length that is sufficient to identify target portions of the genome, as discussed further below. In current practice, the segments of sheared DNA are preferably between 100-600 base pairs (“bps”) in length, more preferably 150-500 bps in length, still more preferably 200-500 bps in length, even more preferably 250-450 bps in length, most preferably about 300 — about 400 bps in length, where “about” means ±25 bps.

In preferred embodiments, the sheared DNA fragments are then tagged in a manner that allows sequences of interest to be captured or otherwise enriched, while allowing non-target sequences to be eluted or otherwise removed from the sequences to be sequenced. The capturing is typically conducted using DNA or RNA sequences complementary to the sequence or sequences of interest (sometimes referred to herein as the “target” sequences).

For example, the sheared fragments can be contacted with SURESELECT® probes (Agilent Technologies, Inc., Santa Clara, Calif.), which are complementary to one or more sequences of interest, such as the 5′UTR of L1. Agilent's “SureDesign” system allows constructing probes customized for target sequences of interest. SURESELECT® probes are biotinylated. The probes are placed in contact with the sheared DNA fragments under conditions allowing them to hybridize to the complementary target sheared DNA sequences with the desired degree of stringency. The sequences hybridized to the probes are then captured by contacting the hybridized sequences with the biotinylated probes to streptavidin-coated magnetic beads. The streptavidin-coated magnetic beads, with the captured sheared DNA sequences hybridized to the probes, can then be retained, while sheared DNA not having a target sequence complementary to that of the probes can be eluted or otherwise removed.

In some embodiments, the streptavidin-coated magnetic beads may be disposed on a solid support. A variety of such solid supports are used in the art but, by way of example, the solid support may be in the form of beads, of a microwell in a multi-well plate, or a slide. In these embodiments, the captured sequences will be retained on the solid support, while sheared DNA that has not hybridized is washed away.

The sheared DNA that hybridized to the probes is then released from the probes, eluted, and subjected to next generation sequencing protocols. The sequences of the sheared DNA are then entered into and analyzed by bioinformatics software programmed to make the determinations discussed below.

### Probes, Sequences, and Methods of Detecting the Number and Locations of pL1s in a Individual Subject

As noted above, over 800 genomic locations have been identified at which a pL1 has been found to insert. The over 800 known pL1 insertion sites known as of this writing are set forth in Table 2, along with some insertion points of fixed L1s that are known to cause mutations in certain cancer types (the fixed L1 insertion points are designated by being preceded by an asterisk). In some embodiments, the inventive methods and devices comprise two types of DNA or RNA probes, which will be discussed in turn.

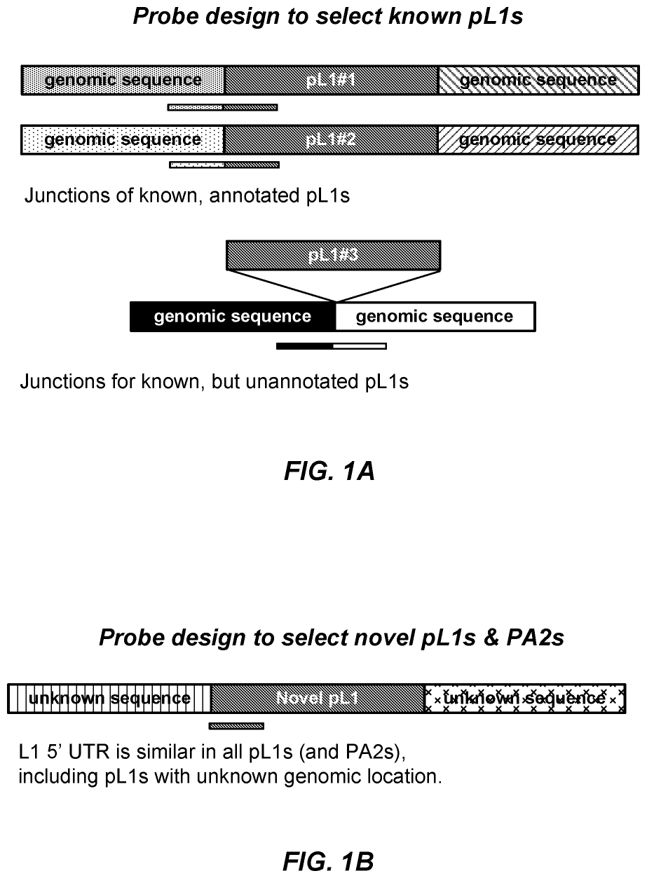

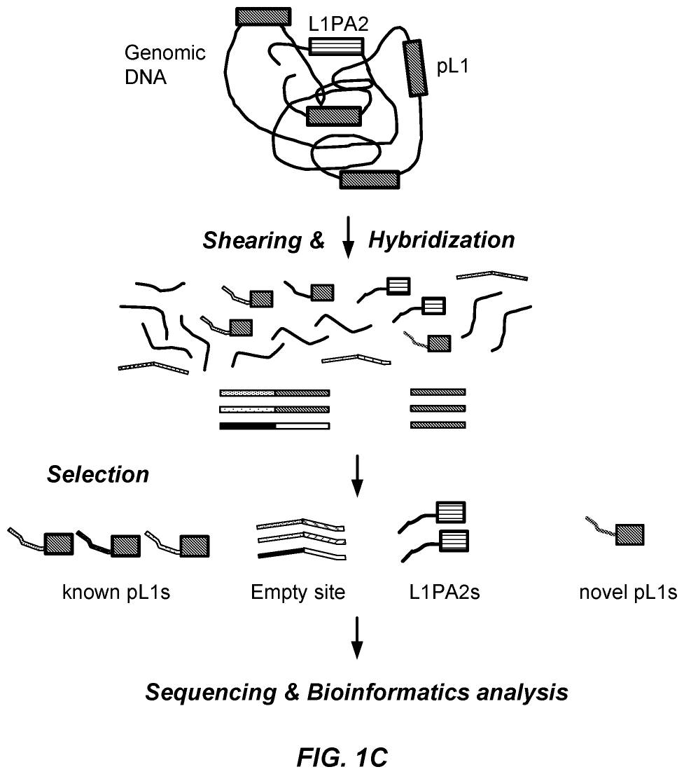

The following discussion sets forth the design of probes to determine, first, which of the over 800 sites at which pL1s are known to insert are occupied by a functional pL1 in an individual, and, second, to determine if that individual also has a pL1 present at a site which has not previously been identified as one in which a pL1 may insert into the genome. FIGS. 1A and B are schematic diagrams which set forth the design plan for probes for use in some embodiments of the inventive methods to pull pL1s and PA2s out of an individual's genome for analysis. FIG. 1C is a schematic diagram of methods used in various embodiments to use probes to detect the locations at which pL1s have inserted into the genome of an individual. For example, if the probes used include probes specific for each one of the over 800 sites at which pL1s have been found to insert in the human genome, and probes that will detect the presence of any pL1s that have inserted at one or more sites at which pL1s are not known to insert as of the time the probe set is used, the methods should result in detecting all of the pL1s in the individual's genome. If the probes used are limited to those for a specific subset of locations at which pL1s are known to insert, such as a probe set designed to detect the presence of pL1s at the specific locations shown in Table 2 to be present only in persons who have been diagnosed with specific conditions, then those embodiments will detect only the presence of pL1s at those positions as opposed to all of the positions in the individual's genome at which a pL1 has inserted. For clarity, it is noted that an assay using probes for all of the sites at which pL1s are known to insert will also detect pL1s that have inserted at sites identified in Table 2 as present only in persons with specific conditions. Using a probe set specific to one or more chosen subsets, however, such as to detect whether an individual has pL1s inserted at one or more sites that are occupied by a pL1 only in individuals that have been diagnosed with prostate cancer, allows gathering desired information while sharply reducing the number of genomic fragments that have to be sequenced, and the resulting number of sequences that have to be analyzed by bioinformatics, and thus can reduce the cost of reagents used and time to analyze and report the information of interest.

FIG. 1A, top two lines, shows the design of probes to detect the presence of pL1s that insert at known, annotated locations in the genome (pL1#1 and pL1#2 in FIG. 1A). Probes designed to detect whether the genome of an individual does or does not have a pL1 inserted at one of the over 800 sites at which pL1s are known to insert consist of a DNA sequence with two components: (1) a sequence complementary to the genomic sequence immediately before the site at which a pL1 is known to insert, followed without interruption by (2) a sequence complementary to the pL1 5′ UTR. Design of such probes is discussed in more detail later in this section.

FIG. 1A also shows the design of a probe for a site at which a pL1 is known to insert, but which has not yet been annotated in the consensus human genome. The presence of such known, but unannotated pL1s, can be detected using probes similar to those just described to detect pL1# and pL1#2. Probes such as those shown for pL1#1 and 2 for pL1s at sites that have not yet been annotated, however, are more costly than probes for sites that have been annotated. The probe shown for pL1#3 avoids the cost concern by being comprised of the genomic sequence at the point the unannotated pL1 has been found to insert. For clarity, it is noted that all functional pL1s have the same or closely similar sequences. The designations of the pL1s that insert at the particular hypothetical sites shown in FIG. 1A as pL1s “#1,” “#2,” and “#3” is for the reader's convenience of reference regarding the sites at which a pL1 is known to insert into the genome, and in determining whether a pL1 has or has not inserted at any particular site in the genome of a particular individual or a particular population of individuals.

Turning now to designing probes for use in various embodiments of the inventive methods, the first type of probes consists of sequences designed to be complementary to the sequences surrounding and including a plurality of, and preferably all, of the known pL1 insertion points (that is, a first probe has a sequence designed to be complementary to the first pL1 insertion site known on chromosome 1, a second probe has a sequence designed to be complementary to the second pL1 insertion site known on chromosome 1, and so on). The sequences are preferably short enough to be readily made, but long enough to hybridize to and thereby capture segments of sheared DNA that are complementary to the probe under the selected hybridization conditions. Analyzing the fragmented DNA captured by the probes then reveals whether the subject has a pL1 inserted at each of the insertion points for which a probe is provided, whether the pL1 is inserted in each of the two copies of the chromosome in the subject's genome or just one, and whether the pL1 is likely to be full-length and therefore capable of being active, in which case the practitioner can optionally choose it as a candidate for further sequencing to verify its sequence and if the pL1 is indeed full-length.

For each of the pL1s annotated in the human genome, the selection of the coordinates should account for the presence of the respective L1s in the genome. For such pL1s, the human genome sequence immediately upstream or immediately downstream, or both, of the location of the pL1 insertion point will be used to determine the complementary sequences to be used for the probes. (Probes to the genomic sequence either upstream or downstream of an insertion point are expected to capture sequence from an inserted pL1; having probes to the genomic sequence both upstream and downstream of an insertion point provides redundancy and can be used in some embodiments.)

To illustrate how the probes allow determining which pL1s are present in an individual and whether the individual has a pL1 on one copy or of both of a particular chromosome at a particular insertion point, the discussion below uses as an example the first pL1 insertion point in chromosome 1. Referring to Table 2, the first pL1 insertion point shown on chromosome 1 is at position 32004332. If the practitioner elects, as an example, to use DNA probes of 300 bp 5′ and 300 bp 3′ of the insertion point, the probes will therefore be made to have a sequence complementary to that of chromosome 1 from position 32004032 to position 32004632. (For the reader's convenience in focusing on the positions of the genomic sequence of interest, some of the discussion below omits the leading numbers 32004, which are indicated by an apostrophe.)

When fragmented DNA from whom the DNA sample is captured, eluted, and sequenced, if the individual has no pL1 present at this site on chromosome 1, all the DNA captured by the probes for this pL1 insertion point on chromosome 1 will have the normal genomic sequence at positions '032 to '632 (sites at which a pL1 has not inserted in a subject's genome are sometimes referred to as an “empty site”). If a pL1 is present on one of the two copies of the chromosome at position '332, sequencing of the sheared DNA captured by the DNA probe will reveal (1) some sequences that have the normal genomic sequence at positions '032 to '632 and some that have the normal genomic sequence at positions '032 to '332 and then a portion of sequence from pL1 and (2) some sequences that have pL1 sequence, followed by the normal genomic sequence of positions '333 to '632. If the subject has a pL1 present on both copies of chromosome 1, sequencing of the sheared DNA captured by the DNA probe will determine that all the sequences have the normal genomic sequence at positions '032 to '332 and then a sequence from pL1, and other sequences having a portion of pL1 sequence, followed by the normal genomic sequence of positions '333 to '632.

As persons of skill are aware, pL1 can insert into the genome in either 5′ to 3′ orientation or 3′ to 5′ orientation. As only full-length pL1 can be active, if the 5′ sequence of the pL1 does not commence with the start of the pL1 5′ UTR, in whichever orientation the pL1 has inserted, the pL1 cannot be full length, and cannot be active. Similarly, for sequences having a 3′ portion of pL1, if the pL1 sequence does not terminate in the end of the pL1 3′UTR, the pL1 cannot be full length, and cannot be active. The genomic sequences at locations in which insertions of pL1 have occurred that are less than full length can optionally be reviewed to determine whether the insertion of L1 sequence has disrupted a coding sequence, has disrupted a promoter, or might otherwise be causative of a disease or contribute to disease progression. Only pL1s that commence with the beginning of the pL1 5′ UTR and end with the end of the 3′ UTR can be full length and are likely to have the capacity to be functional. Thus, the sequencing allows a ready determination of whether the pL1 present is likely to be full-length, and therefore has the capability to generate de novo inserts or other types of genomic instability associated with L1 enzymatic function.

A second type of probe, a sequence complementary to the beginning of the L1 5′UTR is also present on the solid support or supports. Preferably, this second type of probe comprises a sequence of about 300 bp to about 400 bp of the beginning of the 5′UTR, with about here meaning ±25 bp. The L1 5′UTR is approximately 900 bp in length, but the probes use a sequence complementary to that of the beginning of the 5′UTR sequence as, once again, only full-length L1s that might be active are of interest. These probes, which for convenience may be referred to as the “L1 probes” will hybridize to the 5′UTR of any full-length L1 present in the sample, including known L1s and any unknown L1s that are present in the sheared DNA, along with any genomic sequence upstream of the pL1 that is on the segment of sheared DNA.

Sequencing of the DNA sequences upstream of the L1s captured from the subject's sample and comparing those sequences to the sequences upstream and downstream of the 826 sites at which pL1 is known to insert, and all full-length L1s annotated in the human genome will reveal whether each sequence captured by the L1 probe is (1) from one of the already identified pL1 insertion points, (2) from the site of a previously annotated fixed L1 or, (3) a site not previously identified as a L1 insertion point and therefore a previously unknown pL1.

Further, the L1 probe acts as an internal control to confirm that all components of the method worked as intended. If the methods and devices are working as intended, the pL1 probe will capture all the full-length L1s present in the individual's genome, including not only the polymorphic L1s, which by definition can vary in number from individual to individual, but also the fixed L1s, which by definition are the same in every individual. Specifically, as graphically depicted in FIG. 1C, the probe detects the presence of the fixed L1 type referred to as “PA2.” While in some embodiments, the inventive methods can seek to detect the presence of all fixed L1s in the genome, the detection of PA2s is preferred. First, this reduces the number of fixed L1s to be sequenced and analyzed to a little under 1000 per genome. Second, the sequence of PA2 is closer to that of pL1s than the sequence of other fixed L1s, and thus serves as a better control for determining if the sample has been handled correctly such that the number of pL1s detected is reliable, as discussed in the next paragraph.

A number of factors can affect whether the inventive methods work as intended, or whether they are providing inaccurate results due to mishandling of the sample or other procedural problems. For example, assume the DNA in the sample has degraded due to improper storage prior to the hybridization step or the wash buffers have been prepared with incorrect salt concentrations. In such cases, L1 sequences in the sample may not hybridize to the L1 probes or may wash off the L1 probes prior to the elution step. Since the genomic sequence upstream and downstream of each fixed L1 is known, a comparison of the readout of sequences of genomic DNA around the fixed L1s to the sequences of genomic DNA around the L1s in the sample allows the practitioner to determine the percentage of the annotated fixed L1s detected in each sample compared to the number known to be present in the human genome. As persons of skill are aware, some of the fixed L1s are located in regions of the genome with repetitive sequences and in some cases, the repetitive nature of the sequences surrounding the L1s makes it difficult to distinguish one of these fixed L1s from another. Accordingly, it is expected that, when the methods work as intended, the presence of approximately 97% of the almost 1000 annotated PA2s should be detected. Detection of less than 95% of these annotated fixed L1s indicates that there has been a problem with the assay. In such cases, the practitioner can review the sample to determine if the problem is with the quality of the DNA, in which case a fresh DNA preparation should be used, or if there was a problem with preparation of the reagents, in which case fresh reagents should be prepared and the test rerun using the fresh reagents.

The sections below discuss various embodiments of the inventive methods and devices.

### Isolation of Genomic DNA and Shearing

As mentioned, in some embodiments, the inventive methods involve isolating DNA from a subject and hybridizing it to probes. Obtaining DNA from a subject is well known, as evidenced by the kits provided at modest cost by companies which offer DNA analysis to members of the public. Isolating DNA and sequencing it has been well known in the art for decades, as exemplified by Roe, Crabtree, and Khan, DNA ISOLATION AND SEQUENCING, John Wiley & Sons, New York, 1996. Kits and equipment for isolation of research-ready genomic DNA are commercially available, as exemplified by the GenFind V3 Blood and Serum DNA isolation Kit (Beckman Coulter Life Sciences, Indianapolis, Ind.), which can be performed using a 96-well plate configuration to increase sample throughput. A Biomek i7 Hybrid Genomics workstation (Beckman Coulter Life Sciences) can be used for automated processing of 96 samples at a time. It is assumed that the practitioner is familiar with methods for isolating genomic DNA suitable for use in the inventive methods and systems.

Shearing and other methods for randomly fragmenting DNA have been used since the 1970s, and one of the present inventors was one of the originators of DNA shearing in the preparation of DNA sequencing libraries. See, Deininger, Anal Biochem, 1983, 129(1):216-223. Low pressure shearing as a technique for obtaining randomly fragmented DNA was investigated as early as 1990 (see, e.g., Schriefer et al., Nucleic Acids Res. 1990; 18(24):7455-7456). Hydrodynamic shearing of DNA was widely adopted in the 1990s and 2000s, as discussed in, e.g., Thorstenson et al., Genome Res., 1998; 8:848-855; doi:10.1101/gr.8.8.848; Oefner et al., Nucleic Acids Res., 1996, 24:3879-3886; Hengen, Trends Biochem Sci, 1997, 22(7):273-274; and Joneja and Huang, Biotechniques, 2009, 46(7):553-556. More recent techniques for fragmenting DNA include lateral cavity acoustic transducers (LCATs) designed by Okabe and Lee (J Laboratory Automation, 2014, 19(2):163-170) that can be integrated into microfluidic platforms to automate DNA processing. Okabe and Lee note that it is desirable to fragment the DNA to about the size of the probes to improve both hybridization and sensitivity. Id. It is assumed that the practitioner is familiar with the various methods known in the art for fragmenting DNA, whether by shearing or another method, to sizes desired by the practitioner for use in the methods disclosed herein.

### Probes

DNA or RNA probes are used to capture complementary DNA from the subject. As discussed above, the compositions and methods comprise two types of DNA or RNA probes: a first set of probes which are complementary to the genomic DNA at the sites in the genome at which pL1s are known to insert, and a second probe which is complementary to 200 or more bases of L1 sequence, preferably the first 200 or more bases of the beginning of the 5′UTR. Current technology makes it relatively convenient to make probes of about 300-about 400 bases, with “about” meaning ±25 bases, and to sequence DNA of about that length that hybridizes to those probes. Table 2 sets forth the insertion points of the over 800 sites at which pL1s are known as of this writing to insert in the genome. A probe consisting of a sequence complementary to the 300 bases upstream of the pL1 insertion point for any given known pL1 insertion point is expected to hybridize uniquely to sheared DNA from the subject from that genomic position which, depending on where the subject's DNA sheared randomly, may also carry with it L1 sequence from the 3′ end of the L1 or the beginning of the L1 5′UTR, if a full-length pL1 is present in the subject at that site. Similarly, a probe consisting of a sequence complementary to the 300 bases downstream of the pL1 insertion point for any given known pL1 insertion point is expected to hybridize uniquely to sheared DNA from the subject from that genomic position which, depending on where the subject's DNA sheared randomly, may also carry with it L1 sequence from the end of the L1 3′UTR, if a full-length pL1 is present in the subject at that position.

As practitioners will recognize, DNA and RNA synthesis and DNA sequencing technologies are continually improving and the cost and difficulty of synthesizing longer probes is expected to come down. The use of longer probes, such as probes between about 400 and about 500 bases in length, between about 500 and about 600 bases in length, between about 600 and about 700 bases in length, between about 700 and about 800 bases in length, between about 800 and about 900 bases in length, or between about 900 and about 1000 bases in length are expected to be useful in the compositions and methods as the cost and ease of sequencing makes them cost effective, with “about” meaning ±25 bases. Probes longer than 1000 bases could be used if price and synthesis difficulty come down enough to justify their use, but are believed to be unnecessary, as they are not expected to improve the ability of the compositions and methods to identify the presence of pL1s in the subject over probes of between about 200 to about 1000 bases in length.

As noted in the Okabe and Lee reference cited in the preceding section, the lengths of the probes and of the sheared DNA from the subject are preferably about the same length. Thus, if the practitioner uses a longer probe, the DNA of the subject is preferably sheared to a similar length. It is expected that it is within the skill of the practitioner to adjust the shearing techniques used to shear DNA samples to desired lengths, such as those mentioned above.

### Modification of Probes, Hybridization, and Capture of Targeted DNA Solid Supports

The inventive methods, systems, and apparatuses can use DNA or RNA probes attached to supports to capture for analysis DNA from the subject. Synthesizing DNA or RNA sequences for use as probes and attaching them to supports, or synthesizing DNA or RNA probes directly on a solid support has been known in the art for at least two decades. For example, the Affymetrix, Inc. GENECHIP®, has been sold commercially since 1994.

DNA or RNA probes can be synthesized with terminal modifications that allow them to attach to glass or other surfaces, while still being able to hybridize to target sequences. Various options are available in the art for capture and enrichment of the target DNA sequences using probes attached to solid supports. One example is the Agilent SureSelect^{XT HS} target enrichment system discussed above, in which the probes are biotinylated and captured by magnetic beads coated with streptavidin. Another technique attaches DNA to a glass surface by attaching a digoxigenin (dig) molecule to the DNA and attaching an anti-dig antibody to the glass surface by non-specific adsorption. The DNA molecule is then tethered to the glass surface by allowing the dig to be bound by the anti-dig antibody. See, e.g, Kruithof et al., Nat Struct Mol Biol. 2009; 16(5):534-40; Smith et al., Science. 1992; 258:1122-1126. For convenience of reference, modifications of DNA or RNA probes that allow the probes to specifically bind to a capture molecule disposed on a solid support may be referred to herein as being “tags” and probes bearing such modifications as being “tagged.” When targeted DNA hybridizes to the tagged probes, the hybridized DNA can then be captured on the solid supports, allowing the DNA which has not hybridized to the probes to be eluted, thereby enriching the targeted DNA.

Glass or silica can be treated with amino silane reagent to coat their surfaces with amines or epoxides, which can then react with modified nucleotides to bind DNA to the surface. Schlingman et al., Colloids Surf B Biointerfaces. 2011; 83(1): 91-95, disclose a method to attach DNA to a glass surface using N-hydroxysuccinimide (NHS) modified PEG. The glass surface is coated with silane-PEG-NHS and DNA of interest is modified with a single terminal amine group that allows covalent linkage through a reaction between the NHS group and the amine Adessi et al., Nuc Acids Res, 2000; 28(20) p. e87, doi.org/10.1093/nar/28.20.e87, review a variety of chemistries that have been used to covalently attach DNA to glass or other surfaces, including 5′-succinylated target oligonucleotides immobilized on amino-derivatised glass slides, 5′-disulfide modified oligonucleotides bound via disulfide bonds onto thiol-derivatised glass slides, the use of cross-linkers, such as phenyldiisothiocyanate or maleic anhydride, and the use of 1-ethyl-3-(3-dimethylaminopropyl)-carbodiimide hydrochloride (EDC). Adessi et al., also note that carbodiimide chemistry has been used with supports such as amino controlled-pore glass, latex beads, dextran supports, and polystyrene microwells. It is assumed that the practitioner is familiar with these and other methods of using DNA and RNA probes to capture and enrich from a genome target DNA sequences for sequencing.

Many conventional chips for capturing DNA are microarrays, in which the positions of the probes are registered and the information desired by the practitioner is the presence or absence of DNA hybridized to the probe at a particular location or plurality of locations. In embodiments of the inventive methods and systems, however, the information of interest is the sequence of the segments of sheared DNA from the subject. Thus, it is unnecessary to have the probes at particular positions on the solid surface. Accordingly, while the solid support can be a planar surface, such as a slide or a chip, it can alternatively be a bead or a well in a multi-well plate.

In some embodiments, fragmented or sheared DNA from the subject is hybridized to complementary DNA or RNA probes to capture DNA of interest. Protocols and conditions for hybridizing fragmented or sheared DNA to probes are well known in the art and it is expected that practitioners are familiar with the guidance already available in this area.

As practitioners will appreciate, the sequences used as probes for the subject's genomic DNA are based on consensus sequences in GenBank. The sequences of subjects are expected to contain variations from the consensus sequence, due to single nucleotide polymorphisms (SNPs) or to other genetic variations. It is expected that the hybridization conditions will not be so stringent as to prevent hybridization due to these variations. Similarly, it is expected that the 5′UTR of functional pL1s will contain occasional SNPs or other genetic variations. It is expected that the practitioner can readily select hybridization conditions that will not be so stringent as to prevent hybridization due to these variations. It is noted that adjusting stringency conditions to allow desired hybridization is routine in the art.

### Release and Hybridization

Once the targeted DNA has hybridized to the probes and captured on the solid supports, non-hybridized DNA is typically eluted, as in standard protocols for enrichment of target DNA. The targeted DNA that has hybridized to the probes is then released, eluted, amplified, and provided to conventional DNA sequencing. The amplification can be by any convenient means deemed suitable by the practitioner, including conventional PCR or droplet digital PCR (see, e.g. Olmedillas-Lopez et al., Mol Diagn Ther. 2017 October;21(5):493-510. doi: 10.1007/s40291-017-0278-8. As tens of thousands to millions of fragments of targeted DNA are typically captured in such protocols, the sequencing typically results in a like number of sequences. The sequences of the targeted DNA are typically then entered into a bioinformatics program which analyzes the sequences.

### Bioinformatics Programs

In some embodiments, DNA from is sequenced and analyzed to detect with respect to each of the over 800 sites at which pL1 is known to insert into the genome, to determine which sites in an individual's genome have a pL1 present, to detect the presence of fixed L1s in the subject's genome, and to detect the presence of any pL1s in the subject at sites at which a pL1 was not previously known to occur. The detection of these L1 elements is developed from analyzing normal, non-L1 element genomic sequence that hybridizes to the DNA or RNA probes. Given the large number of genomic sequences (over 800 just for the pL1 elements identified as of this writing), to be analyzed to determine the particular sites a pL1 has inserted in the subject's genome, plus the determination of whether all the approximately 1000 fixed L1PA2 elements have been detected, to verify that the hybridization and other conditions worked as intended, it is not possible for these analyses to be performed by hand calculation. Accordingly, performing the methods of the invention requires the use of bioinformatics software to analyze the sequence information.

Dozens of free and paid bioinformatics programs are available for comparing and analyzing nucleotide sequences. To list just a few, the free software programs include the European Molecular Biology Open Software Suite (“EMBOSS”), Integrated Genome Browser (IGB), GENtle, and Jalview. Paid DNA bioinformatics software includes CLC Genomics Workbench (QIAGEN Aarhus, Aarhus, Denmark), Partek® Genomics Suite, and Vector NTI Advance® (Invitrogen). Practitioners typically have preferences based on their prior use of and familiarity with particular software packages and compatibility with their computer system. It is anticipated that the practitioner is capable of choosing and using a software package suitable for use in the inventive methods and systems.

### Systems, Devices, and Kits

As discussed above, in some embodiments, two sets of DNA or RNA probes, or combinations of DNA and RNA probes, are used, a first set which is complementary to genomic sections in which pL1s have previously been found in the human genome, and a second set, which is complementary to several hundred bases of the sequence of L1, preferably of the beginning of the L1 5′UTR or the end of the 3′ UTR (these sets of probes are sometimes referred to herein as the “first set” and the “second set” of probes, respectively, or together simply as “the probes”). The probes are preferably disposed on solid supports. In some embodiments, the probes are covalently attached to the supports so they do not wash off the supports in later wash and elution steps. In some embodiments, the probes are conjugated or fused to provide a terminal modification, or tag, which allow the probes to specifically bind to a solid support, either directly, or through a linker that specifically binds the tag. For example, the probes may be modified by biotinylation or by containing digoxigenin, as discussed above in the section on probes. When targeted DNA hybridizes to the tagged probes, the hybridized DNA can then be captured on the solid supports, allowing the DNA which has not hybridized to the probes to be eluted, thereby enriching the targeted DNA.

Isolated DNA from an individual is obtained (the DNA may be obtained in the form of cells which are lysed and from which the DNA is isolated, or may be obtained as already-isolated DNA) and is fragmented into a size selected by the practitioner, typically by shearing (as the DNA is preferably fragmented by shearing, for convenience of reference, the DNA fragments will be referred to below as having been “sheared,” even if another technique has been used to fragment the DNA). The sheared DNA is in lengths of at least 100 bases in length, more preferably about 200 bases, more preferably about 250 bases, still more preferably about 300 to about 400, in some embodiments about 400 to about 500, in some embodiments about 500 to about 600, in some embodiments about 600 to about 700, with “about” in this context meaning ±25 bases. The fragmented DNA from the subject is then placed in contact with the DNA or RNA probes under conditions which allow fragmented DNA from the subject that is complementary to that of the probes to hybridize. The fragmented DNA from the subject which has not hybridized to the DNA or RNA probes is washed away, after which the DNA which has hybridized is eluted and sequenced.

In some preferred embodiments, the process of hybridizing and sequencing the DNA fragments is conducted in an automated device configured for the purpose. In some embodiments, the automated device is a microfluidic device. In some embodiments, the automated device is configured to allow high-throughput of samples. This can be accomplished by, for example, using a multi-well plate system or by other apparatuses allowing multiple runs of samples undergoing the same procedures, such as parallel microfluidic chambers.

The sequencing of the DNA from the subject that has hybridized to the DNA or RNA probes typically results in tens of thousands to millions of sequences from each individual. The sequences are provided to a bioinformatics program which is programmed to compare the sequences from the first set of probes to the sequences of the genome upstream and downstream of the insertion points of the 826 sites at which pL1 elements are known to insert into the genome, as listed in Table 2, below, as well as the L1 5′UTR and '3 UTR sequences and to identify and record (a) whether for each of the known pL1 insertion sites, the individual shows the genomic sequence present at each of the potential pL1 insertion points, (b) whether the sequence shows that the genomic sequence normally at each potential pL1 insertion point has a L1 5′UTR sequence commencing from the beginning of the 5′UTR, (c) whether, for each potential pL1 site that does have a L1 sequence commencing from the beginning of the L1 5′UTR, the site also has a sequence with the ending of the L1 '3UTR, and (d)(i) the total number of L1s sequences that have bound to the second probe set and the surrounding genomic sequence, (ii) determine from comparing the genomic sequences upstream or downstream, or both, of each of the L1 sequences detected to the genomic sequences surrounding each of the fixed L1s in the genome how many of the fixed L1s have been detected, whereby detecting less than 95% of the number of fixed L1s indicates that there was a problem with the detection and that the subject's DNA should be rescreened, and (iii) determine by detecting any L1 elements that are surrounded by genomic sequence not previously identified as a point at which an L1 element inserts that the individual has a previously unidentified pL1. As noted in a previous section, Table 2 includes both the approximately 800 currently known pL1 insertion points and, as a positive control, a small number of insertion locations of fixed pL1s known to be active in particular cancers.

In some embodiments, the invention further provides electronic devices configured for determining how many polymorphic LINE-1 elements (“pLls”) which pL1s have a 5′ untranslated region (“5′UTR”) and a 3′UTR, which 5′UTR begins with a contiguous sequence of at least 300 bases and which 3′UTR terminates in a contiguous sequence of at least 300 bases, are present in genomic DNA of a subject, and at which of the sites at which pL1s are known to insert the pL1s are present in the genomic DNA of the subject. The devices comprise a processor and memory, in which the memory stores computer executable instructions for performing the methods set forth above.

In some embodiments, the invention provides kits. The kits provide sets of probes, which can be for detecting with regard to each of the over 800 insertion sites whether or not a pL1 has inserted in that site with respect to an individual's genome, or for detecting with regard to a subset of sites at which a pL1 is known to insert, such as pL1s identified in Table 2 as found by WGS, SCORE, or both, only in individuals diagnosed with breast cancer, pL1s identified in Table 2 as found by WGS, SCORE, or both, only in individuals diagnosed with prostate cancer, pL1s identified in Table 2 as found by WGS, SCORE, or both, in genomes of both individuals diagnosed with breast cancer and in genomes of individuals diagnosed with prostate cancer, but not in genomes of individuals listed in Table 2, column “Cont-WGS,” pL1s identified in Table 2 as found only in individuals diagnosed with Alzheimer's Disease, or, pL1s identified in Table 2 as found by WGS, SCORE, or both, in individuals diagnosed with Alzheimer's Disease, in individuals diagnosed with breast cancer, and in individuals diagnosed with prostate cancer, but not in genomes of individuals listed in Table 2, column “Cont-WGS.

### EXAMPLES

### Example 1

This Example sets forth materials and methods that were used for finding polymorphic L1s in studies reported herein.

### Materials and Methods

### Human Whole Genome Sequencing Samples:

Human Prostate adenocarcinoma and Breast invasive carcinoma WGS (whole genome sequencing) samples were downloaded from the National Cancer Institute Genomic Data Commons (“GDC”) Data Portal. Human Cognitively normal (control) and Alzheimer's Disease WGS samples were downloaded from the Alzheimer's Disease Neuroimaging Initiative (“ADNI”) data archive. Cognitively normal patients with cancer were excluded from the control group.

### Prostate Cancer Patient Samples

Buffy coats and patient metadata from prostate cancer patients were obtained through the Tulane Urology Department Biospecimen Bank. Genomic DNA was extracted using the DNeasy Blood and Tissue kit (Qiagen N.V., Germantown, Md.) and submitted for SCORE analysis.

### Cell Culture

MCF7 cells (American Type Culture Collection (ATCC), Manassas, Va., #HTB-22) were maintained in Minimum Essential Media (“MEM”) (Gibco™, Thermo Fisher Scientific, Waltham, Mass.) supplemented with 10% bovine serum (Gibco), sodium pyruvate, essential and nonessential amino acids, and L-glutamine.

### Targeted Sequencing

Targeted sequencing probes were designed by Agilent Technologies, Inc. (Santa Clara, Calif.) for all known polymorphic L1 insertion sites and for fixed PA2 loci. Fragmenting of DNA and paired-end sequencing was performed by BGI Americas (San Jose, Calif.).

### Bioinformatic Analysis

Paired-end sequencing files were obtained through SCORE targeted sequencing or extracted from WGS alignment files. The paired alignment files for each sample were aligned separately to the human L1 consensus sequence using STAR v2.3.0e alignment software and allowing one alignment per read (-outFilterMultimapNmax 1) and a maximum of 25 mismatches (-outFilterMismatchNmax 25). Alignments that occurred in the first 700 bp of the

L1 consensus sequence and were in the reverse orientation to L1 were extracted. These reads were then used to find their pair based on matching read IDs. The opposite read pair was then aligned to the human genome using bowtie v0.12.8, requiring unique alignments (-m 1), the tryhard setting (-y), and allowing 3 mismatches (-v 3). Alignments in the resulting file were then parsed for read alignments that occurred within the 5′ upstream region of known polymorphic L1 loci and L1 PA2s. This was done using bedtools v2.22.0.

### Example 2

This Example compares the ability of the inventive methods to detect PA2 fixed L1s to that of whole genome sequencing. PA2s are present in approximately 1000 fixed positions in the genome of all individuals and the number found should therefore be the same regardless of which method is used to detect them.

A study was conducted using information obtained from whole genome sequencing of patient samples diagnosed with Alzheimer's Disease, breast cancer, or prostate cancer, or individuals who had not been diagnosed with any of these conditions (“controls”), and information developed by analyzing the genome of a breast cancer patient and of prostate cancer patients by the inventive methods.

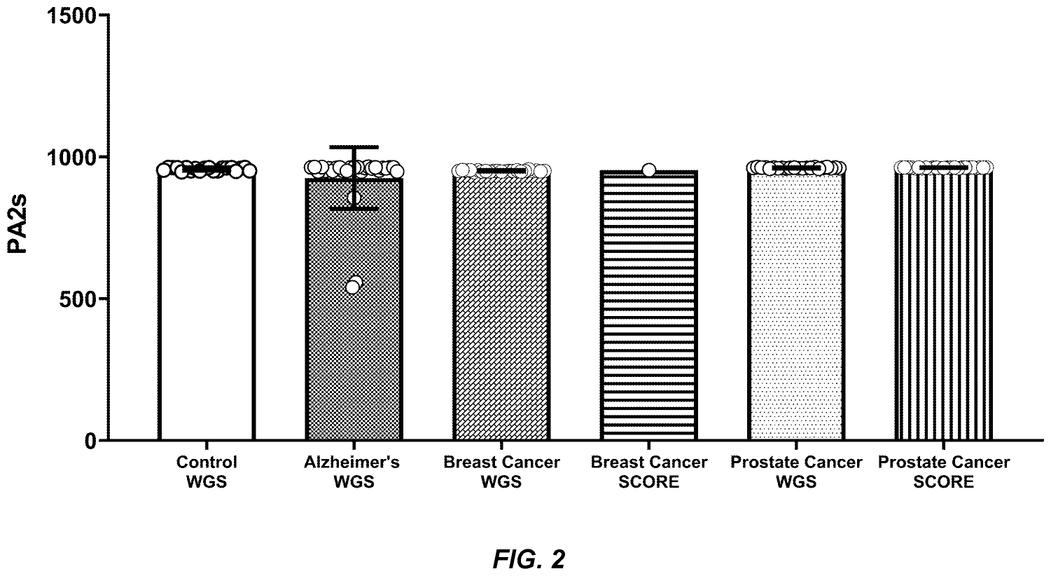

The results of this study are presented as a bar graph in FIG. 2, with each genome analyzed represented as a circle. As shown in FIG. 2, all the genomes analyzed (except for those discussed further below) showed the same number of PA2s. Those genomes analyzed by whole genome sequencing are labeled by the acronym “WGS,” while those analyzed by the inventive methods are labeled with the acronym “SCORE,” which stands for the phrase “Screen for Content Of Retro Elements,” used by the present inventors to refer to some embodiments of the inventive methods.

As noted, two of the genomes in the bar presenting the results for Alzheimer Disease patients whose genomes were analyzed by WGS show just over 500 PA2s in the genomes of those individuals, little more than half the number expected. The results for these individuals show that the whole genome sequencing conducted on the genomes of those two individuals failed to detect hundreds of the fixed L1s actually present, and that there was a problem in the sample preparation or subsequent analysis. Thus, determining the number of PA2s present in a sample acts as a control, in which a result showing the presence of a lower number of fixed L1s than are known to exist indicates that there was a problem with either the sample preparation or the analysis.

FIG. 1B shows a probe that is complementary to the 5′UTR of polymorphic L1s and fixed L1s, but without having a sequence from the genome. DNA fragments from the L1 5′UTR will bind to the probe, and some fragments will include genomic sequence upstream of the 5′UTR. That genomic DNA upstream of the 5′UTR is then sequenced and compared to the consensus genomic DNA and other sources to determine where in the genome the particular 5′UTR is located and to see if the 5′UTR at the genomic location corresponding to the genomic sequence attached to the 5′UTR fragment is the location of a known fixed L1 or a pL1. If a L1 is found to always be present in the consensus genomic DNA or other genome sources at a location that does not correspond to a previously known fixed L1, it can be classified as a previously unknown fixed L1. If a complete L1 sequence is found to sometimes occur at the newly identified location but not always, that would indicate that the probe has detected a pL1 not previously known to occur at the genomic location in question.

### Example 3

This Example shows that the inventive methods detect more pL1s in patient samples than does whole genome sequencing, and is therefore a more sensitive detection method.

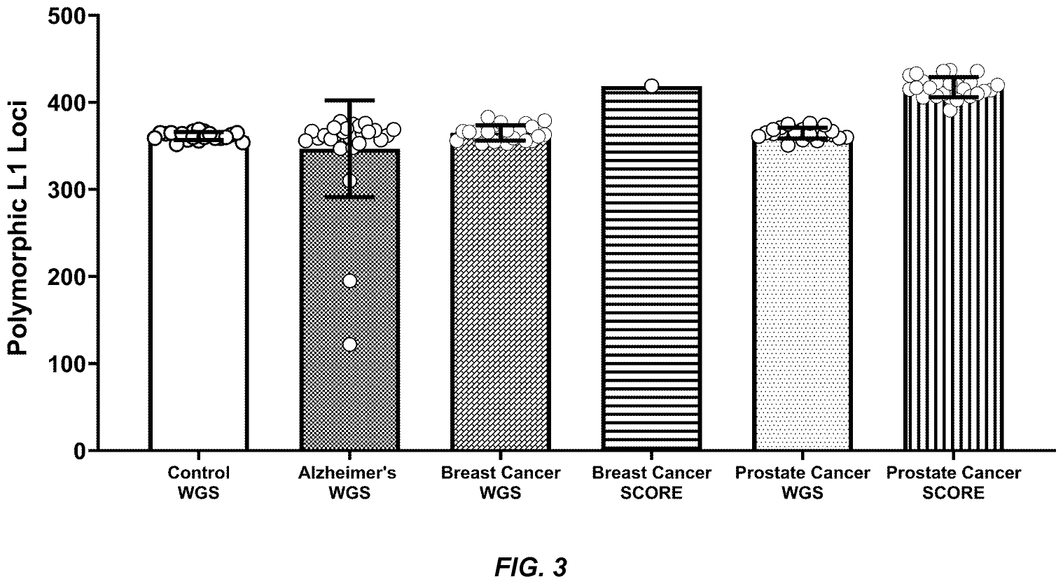

FIG. 3 presents bars representing the number of pL1s found in individuals who had either been diagnosed with one of four conditions, Alzheimer's disease, breast cancer, or prostate cancer, or, as controls, who had not been diagnosed with one of the three conditions mentioned. The genomes indicated in the Figure as having been analyzed by WGS are from the GDS Data Portal and ADNI data archives, as described in Example 1. The genomes of individuals with prostate cancer analyzed by the inventive methods, shown in the bar labeled “Prostate Cancer SCORE,” are from the Tulane Urology Department, as described in Example 1.

The Y axis of FIG. 3 shows the number of polymorphic L1s determined to be present in an individual in one of the four patient or control samples by either whole genome sequencing (“WGS”) or by the inventive methods, referred to on the Figure as “SCORE.” As noted above, SCORE is an acronym for “Screen for Content Of Retro Elements.” The circles at the top of each bar show the number of pL1s found in a sample from a single individual, either with Alzheimer's disease, breast cancer, prostate cancer, or not known to have any of these conditions (“Control”). A sample for one breast cancer patient was available for analysis by the inventive methods and the number of different pL1s found in that patient sample is shown in the bar labeled “Breast Cancer SCORE.” As can be seen from the height of the respective bars, analysis of the genomes of the breast cancer patient and of prostate cancer patients by the inventive methods resulted in detection of notably more pL1s than were detected in patients' cancers of the same organs, but analyzed by conventional by whole genome sequencing. These results demonstrate the increased sensitivity of the inventive methods over the use of a conventional method for finding how many pL1s exist in an individual. Finally, two genomes in the Alzheimer's Disease WGS group showed unusually low numbers of pL1s. These are likely the same two genomes shown in FIG. 2 as not having detected the expected number of PA2s present in the genome, and therefore indicate that there was a problem with those two samples.

### Example 4

This Example describes the results of a study using information developed from genomes of individuals in the GDC Data Portal and the ADNI data archives analyzed by WGS, or from the Tulane Urology Department analyzed by SCORE, as described in Example 1.

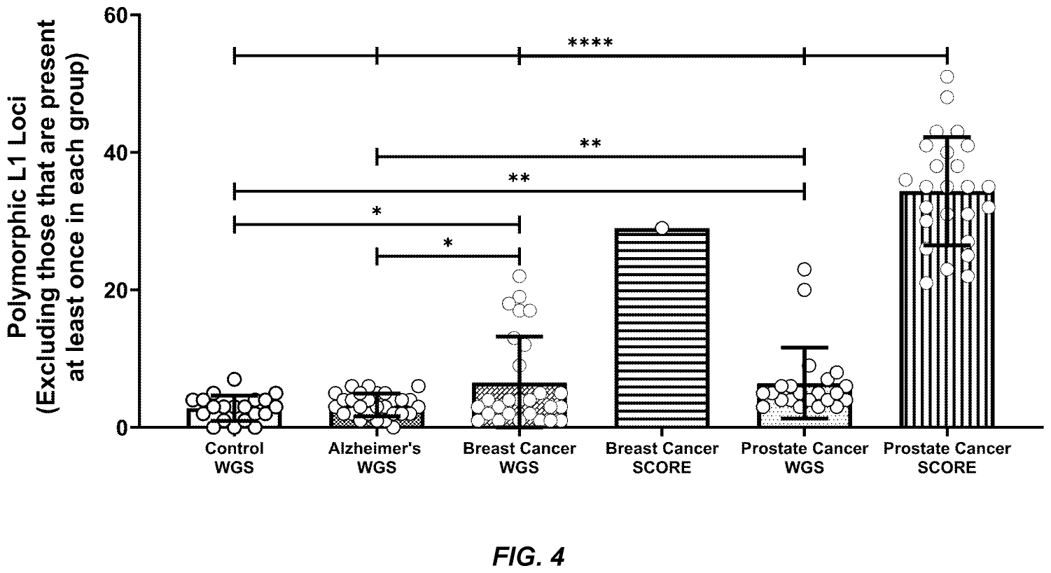

As described elsewhere in this disclosure, genomes differ not only in how many of pL1s have inserted into them, but also in the subsets of the over 800 sites identified to date as to which pL1s insert into the human genome. The locations of each of those individual pL1 insertion sites is set forth in Table 2, below. Individual genomes from individuals that had been analyzed by WGS or by SCORE were examined for the presence of a pL1 at each of the sites identified in Table 2, in an attempt to find pL1s or patterns of pL1s that were markers of either breast or prostate cancer or of Alzheimer's Disease, as an exemplary cognitive disorder. pL1s that were common to at least one individual in every group examined (Alzheimer's, prostate cancer, and breast cancer) were excluded as unlikely to be useful as a marker for any of the particular conditions included in the study. FIG. 4 shows in graphical form the number of pL1s found in each group after excluding pL1s that inserted at sites at which a pL1 was found at least once in each group. As can be seen, the bars reflecting pL1s analyzed by the inventive methods, shown by the label “SCORE” on the Figure, found many more pL1s present in individuals with the conditions in question than did analysis by conventional WGS analysis.

Table 2 lists all of the pL1s identified as of this writing, and identifies the pL1s which were found in genomes of individuals who had developed one of the conditions listed, as well as those present in cognitively normal individuals who had not been diagnosed with cancer (the group labeled as “Control WGS” in Table 2). As can be seen by referring to Table 2, many more pL1s were identified in genomes from prostate cancer patients analyzed by the inventive methods compared to those identified by analysis by WGS.

### Example 5

This Example describes the results of a study using information developed from genomes of individuals in the GDC Data Portal and the ADNI data archives analyzed by WGS, or from the Tulane Urology Department analyzed by SCORE, as described in Example 1.

As noted in the preceding Example, individual genomes from individuals that had been analyzed by WGS or by SCORE were examined for the presence of each of the 826 pL1s identified in Table 2 in an attempt to find pL1s or patterns of pL1s that were markers of either breast or prostate cancer or of Alzheimer's Disease (sometimes abbreviated herein as “AD”), as an exemplary cognitive disorder. pL1s that were common to at least one individual in every group examined (AD, prostate cancer, etc.), were excluded as unlikely to be useful as indicative of increased risk for one of the conditions included in the study. This Example reports the results of the analysis. Table 2 lists 826 known pL1s, and identifies the pL1s which were found in the studies reported here to be present in genomes of individuals who had been diagnosed with AD, breast cancer, or prostate cancer, as well as those pL1s present in individuals who had not been diagnosed with AD, breast cancer, or prostate cancer (for purposes of this study, the last group of individuals were considered to be controls; the column listing the pL1s noted in their genomes is labeled in Table 2 as “Cont WGS.”). Table 2 sets forth for each of the 826 pL1s listed by chromosome and insertion point within that chromosome whether the pL1 was found in the genome of an individual who had been diagnosed with AD, with breast cancer, or with prostate cancer, or who had not been diagnosed with any of these conditions as of the time their genome was analyzed for the presence of pL1s.

FIG. 5 presents the results in graph form. One hundred and sixty-six pL1s were found only in patient with prostate cancer. Referring to FIG. 5, it can be seen that, while 166 pL1s identified were found only in prostate cancer patients in the groups analyzed, additional pL1s were found that were also present in breast cancer patients, but not in AD patients and not in persons that had not been diagnosed with cancer (“Control”). Similarly, additional pL1s were found that were also present in Alzheimer's patients, but not in breast cancer patients or in cognitively normal patients that had not been diagnosed with cancer. Adding these groups together results in 166+80+17+20=283 pL1 loci that were found in persons who had been diagnosed with prostate cancer, but not in the control group. The presence of one or more of the 166 pL1s unique to prostate cancer in an individual's genome is expected to indicate that that individual is at an increased risk of developing prostate cancer compared to the general population.

Referring again to FIG. 5, 80 pL1s were found to be common to both by individuals who had been diagnosed with breast cancer and individuals who had been diagnosed with prostate cancer. Breast cancer is unfortunately common in women. Prostate cancer, of course, occurs only in men. Males do develop breast cancer, but rarely compared to women. Breast cancer and prostate cancer were chosen for analysis in this study in part because the populations affected by the two cancer types rarely overlap. Thus, the finding of 80 pL1s shared by patients diagnosed with one of these cancers suggests that the presence of one or more of these pL1s in an individual's genome indicates that the individual is at heightened risk of developing cancer during their lifetime and should be monitored more closely than might otherwise be the case. Without wishing to be bound by theory, it is believed that the more of these 80 pL1s is present in an individual's genome, the higher the risk that individual will develop cancer during their lifetime compared to those who do not carry those pL1s. If the individual is female, she should be monitored for cancer, and in particular for breast cancer, earlier and more often than if she had no other risk factors. If the individual is a male, he should be monitored more closely for prostate cancer and, with respect to any prostate cancer detected, should be monitored more closely for progression of that cancer, than might be practiced for men without these risk factors.

Ten pL1 loci, identified in Table 2, were found to be unique to breast cancer patients. The presence of one or more of these 10 pL1s in an individual's genome indicates that the individual is at elevated risk of developing breast cancer during their lifetime and should be monitored with breast exams and mammograms earlier than patients without one or more of these pL1s being present. Further, 80 pL1s were found in breast cancer patients that were also present in prostate cancer patients, but not in AD patients or in cognitively normal patients that had not been diagnosed with cancer. Similarly, 17 additional pL1s were found that were also present in Alzheimer's patients and in prostate cancer patients, but not in cognitively normal patients that had not been diagnosed with cancer. Adding these groups together results in 10+80+17+1=108 pL1 loci are unique to breast cancer compared to the control group. The presence of one or more of the 18 pL1s that breast cancer patients share with AD patients indicate that individuals with one or more of those pL1s have an elevated risk of developing breast cancer, Alzheimer's Disease, or both.

Alzheimer's Disease patients were found to have 2 pL1 loci that were not shared with the cognitively normal individuals or individuals with either of the two cancers. The presence of one or both of these 2 pL1s in an individual's genome indicates that the individual is at elevated risk of developing AD during their lifetime and should be monitored for cognitive impairment starting in their early 60s. A number of pL1s found in AD patients were also found in patients with breast cancer, or with prostate cancer. The presence of one or more of these pL1s indicate that those individuals have an elevated risk of developing AD or cancer. For example, 17 pL1s are shared by AD, breast cancer, and prostate cancer patients, thus their presence in a genome would indicate a risk of developing any of these three diseases.

Combined, these findings demonstrate that age and gender should be considered when interpreting pL1 content relevant to the risk of developing disease. This is because females will not develop prostate cancer, while males can, although rarely develop breast cancer. Similarly, defects in DNA repair genes detected by genetic tests combined with pL1 content are expected to have a better predictive power of a disease risk than they do alone.

481 pL1s were found to be shared among the four groups analyzed. A pL1 was considered to be shared if it was found in at least one of the samples within each group. Some of these pL1s could remain important for a specific disease included in this analysis or in other diseases because their allelic frequencies may differ between different groups. For example, some of these pL1s may be found more frequently in breast cancer patients than in controls, which would indicate that they may carry some risk of association with breast cancer. The same is true for any pL1s that are shared between controls and any individual diseases. Table 2 also includes 6 fixed L1s, each set off by an asterisk, which have been found to be active (that is, able to cause mutations) in persons with cancer. As noted earlier, by definition, a fixed L1s is present in every human genome, these six fixed L1s do not by themselves indicate that a person carrying them is at greater risk for cancer than any other member of the population.

TABLE 2

| | insertion | Cont- | AD | BC | BC | PC | PC |
|---|---|---|---|---|---|---|---|
| chrom | site | WGS | WGS | WGS | SCORE | WGS | SCORE |
| chr1 | 32004332 | | 1 | | | | 1 |
| chr1 | 35037706 | 1 | 1 | 1 | 1 | 1 | 1 |
| chr1 | 41502718 | | | 1 | | | 1 |
| chr1 | 48647978 | | | | | | 1 |
| chr1 | 60715678 | | 1 | | | 1 | 1 |
| chr1 | 66030267 | 1 | 1 | 1 | 1 | 1 | 1 |
| chr1 | 67550598 | 1 | 1 | 1 | 1 | 1 | 1 |
| chr1 | 69801765 | | | | | | 1 |
| chr1 | 71019754 | | | | | | 1 |
| chr1 | 71248578 | 1 | | 1 | | | 1 |
| chr1 | 71979421 | 1 | 1 | 1 | 1 | 1 | 1 |
| chr1 | 74984300 | | | | | | |
| chr1 | 81410948 | 1 | 1 | 1 | 1 | 1 | |
| chr1 | 84524089 | 1 | 1 | 1 | | 1 | 1 |
| chr1 | 86214205 | 1 | 1 | 1 | 1 | 1 | 1 |
| chr1 | 86392754 | 1 | 1 | 1 | 1 | 1 | 1 |
| chr1 | 87150794 | 1 | 1 | 1 | 1 | 1 | 1 |
| chr1 | 89783522 | 1 | 1 | 1 | | 1 | 1 |
| chr1 | 95729448 | | 1 | | | | 1 |
| chr1 | 95801827 | 1 | 1 | 1 | 1 | 1 | 1 |
| chr1 | 102568901 | 1 | 1 | 1 | | 1 | 1 |
| chr1 | 105318897 | 1 | 1 | 1 | 1 | 1 | 1 |
| chr1 | 105392486 | 1 | 1 | 1 | 1 | 1 | 1 |
| chr1 | 105857743 | | | | | | 1 |
| chr1 | 105968014 | | 1 | | | | |
| chr1 | 114039827 | 1 | 1 | 1 | 1 | 1 | 1 |
| chr1 | 116084510 | | | | 1 | 1 | 1 |
| chr1 | 116548018 | | 1 | | | | 1 |
| chr1 | 116980814 | | | 1 | | | 1 |
| chr1 | 119394975 | 1 | 1 | 1 | 1 | 1 | 1 |
| chr1 | 121280057 | 1 | 1 | 1 | 1 | 1 | 1 |
| chr1 | 152443884 | | | | | | 1 |
| chr1 | 158696201 | | | 1 | | | 1 |
| chr1 | 158853726 | | | | | | 1 |
| chr1 | 162184333 | | | | | 1 | 1 |
| chr1 | 162610539 | | | | | 1 | 1 |
| chr1 | 163772791 | | 1 | | 1 | | 1 |
| chr1 | 165553135 | 1 | 1 | 1 | 1 | 1 | 1 |
| chr1 | 166218321 | 1 | 1 | 1 | 1 | 1 | 1 |
| chr1 | 166445995 | | | | | | 1 |
| chr1 | 174565515 | 1 | 1 | 1 | 1 | 1 | 1 |
| chr1 | 175389944 | | | | | | 1 |
| chr1 | 179575350 | 1 | 1 | 1 | 1 | 1 | 1 |
| chr1 | 179717828 | | | | | 1 | 1 |
| chr1 | 180841979 | 1 | 1 | 1 | 1 | 1 | 1 |
| chr1 | 184814728 | 1 | 1 | 1 | | 1 | 1 |
| chr1 | 187318924 | 1 | 1 | 1 | 1 | 1 | 1 |
| chr1 | 187497248 | | | | | | 1 |
| chr1 | 187566804 | 1 | 1 | 1 | 1 | 1 | 1 |
| chr1 | 188829883 | | | | | | 1 |
| chr1 | 193549620 | | 1 | | 1 | | 1 |
| chr1 | 193693022 | 1 | 1 | 1 | 1 | 1 | 1 |
| chr1 | 196194529 | 1 | 1 | 1 | 1 | 1 | 1 |
| chr1 | 197676847 | 1 | 1 | 1 | 1 | 1 | 1 |
| chr1 | 201918258 | | | | | | |
| chr1 | 202496193 | | | | | 1 | 1 |
| chr1 | 204822266 | | | | | | 1 |
| chr1 | 210093166 | 1 | 1 | 1 | 1 | 1 | 1 |
| chr1 | 210245871 | | | | | | 1 |
| chr1 | 213530477 | | | | 1 | | 1 |
| chr1 | 215716023 | | | | | | 1 |
| chr1 | 218188594 | 1 | 1 | 1 | 1 | 1 | 1 |
| chr1 | 222579096 | | | 1 | | | 1 |
| chr1 | 225393576 | | | | | | 1 |
| chr1 | 237238565 | 1 | 1 | 1 | 1 | 1 | 1 |
| chr1 | 239792823 | 1 | 1 | 1 | 1 | 1 | 1 |
| chr1 | 242311549 | 1 | 1 | 1 | | 1 | 1 |
| chr1 | 247850462 | 1 | 1 | 1 | | 1 | 1 |
| chr1 | 248057638 | 1 | 1 | 1 | | 1 | 1 |
| chr2 | 4781322 | 1 | 1 | 1 | 1 | 1 | 1 |
| chr2 | 11142259 | 1 | 1 | 1 | 1 | 1 | 1 |
| chr2 | 16781023 | 1 | 1 | 1 | 1 | 1 | 1 |
| chr2 | 23190984 | 1 | 1 | 1 | 1 | 1 | 1 |
| chr2 | 29645153 | | | 1 | | | 1 |
| chr2 | 35879328 | 1 | 1 | 1 | | 1 | 1 |
| chr2 | 35879337 | 1 | 1 | 1 | | 1 | 1 |
| chr2 | 36345677 | 1 | 1 | 1 | 1 | 1 | 1 |
| chr2 | 36570229 | 1 | 1 | 1 | 1 | 1 | 1 |
| chr2 | 36619715 | | | | 1 | | 1 |
| chr2 | 41913656 | 1 | 1 | 1 | 1 | | 1 |
| chr2 | 42051389 | 1 | 1 | 1 | | 1 | 1 |
| chr2 | 43893639 | 1 | 1 | 1 | 1 | 1 | 1 |
| chr2 | 49165205 | | | | | | 1 |
| chr2 | 53460065 | | | 1 | | | 1 |
| chr2 | 57521044 | | | | 1 | | |
| chr2 | 63593495 | | | | | 1 | 1 |
| chr2 | 68049176 | | 1 | 1 | | | |
| chr2 | 69168887 | | 1 | 1 | | 1 | 1 |
| chr2 | 71644631 | 1 | 1 | 1 | 1 | 1 | 1 |
| chr2 | 79929362 | | | 1 | | 1 | 1 |
| chr2 | 82098017 | 1 | 1 | 1 | 1 | 1 | 1 |
| chr2 | 86888391 | 1 | 1 | 1 | 1 | 1 | 1 |
| chr2 | 100310639 | | | | | 1 | 1 |
| chr2 | 102912441 | 1 | 1 | 1 | 1 | 1 | 1 |
| chr2 | 103188842 | 1 | 1 | 1 | 1 | 1 | 1 |
| chr2 | 106216638 | | | 1 | | | 1 |
| chr2 | 107997801 | | | | | | 1 |
| chr2 | 109450667 | | | 1 | | 1 | 1 |
| chr2 | 109459544 | | | | | | 1 |
| chr2 | 113267420 | 1 | 1 | 1 | 1 | 1 | 1 |
| chr2 | 114826303 | | | | | | 1 |
| chr2 | 119659368 | 1 | 1 | 1 | 1 | 1 | 1 |
| chr2 | 120295108 | | | 1 | | | |
| chr2 | 124772475 | | | | | | 1 |
| chr2 | 126941639 | 1 | 1 | 1 | 1 | 1 | 1 |
| chr2 | 129372165 | | | 1 | | 1 | 1 |
| chr2 | 129573941 | 1 | 1 | 1 | | 1 | 1 |
| chr2 | 132769079 | 1 | 1 | 1 | 1 | 1 | 1 |
| chr2 | 133867344 | | | | | | 1 |
| chr2 | 135117710 | | | | | | 1 |
| chr2 | 144010765 | 1 | 1 | 1 | 1 | 1 | 1 |
| chr2 | 144304962 | | | | 1 | 1 | 1 |
| chr2 | 144618526 | 1 | 1 | 1 | 1 | 1 | 1 |
| chr2 | 144626224 | | | | | | 1 |
| chr2 | 149814472 | | | | | | 1 |
| chr2 | 150518957 | | 1 | | | 1 | 1 |
| chr2 | 151744032 | | | 1 | 1 | | 1 |
| chr2 | 153870310 | 1 | 1 | 1 | 1 | 1 | 1 |
| chr2 | 156527796 | 1 | 1 | 1 | | 1 | 1 |
| chr2 | 157907605 | | | | | | 1 |
| chr2 | 158422866 | 1 | 1 | 1 | 1 | 1 | 1 |
| chr2 | 160971028 | 1 | 1 | 1 | 1 | 1 | 1 |
| chr2 | 162748906 | | 1 | | | | |
| chr2 | 164288494 | | | | | | 1 |
| chr2 | 165525734 | | | | | | 1 |
| chr2 | 167844965 | 1 | 1 | 1 | 1 | 1 | 1 |
| chr2 | 170105115 | 1 | 1 | 1 | 1 | 1 | 1 |
| chr2 | 173179985 | 1 | 1 | 1 | 1 | 1 | 1 |
| chr2 | 176352722 | 1 | 1 | 1 | 1 | 1 | 1 |
| chr2 | 177983639 | | | | | | 1 |
| chr2 | 178837804 | 1 | 1 | 1 | 1 | 1 | 1 |
| chr2 | 181704414 | 1 | 1 | 1 | 1 | 1 | 1 |
| chr2 | 191478683 | 1 | 1 | 1 | | 1 | 1 |
| chr2 | 194083171 | 1 | 1 | 1 | 1 | 1 | 1 |
| chr2 | 197770314 | 1 | 1 | 1 | 1 | 1 | 1 |
| chr2 | 198505485 | 1 | 1 | 1 | 1 | 1 | 1 |
| chr2 | 199779883 | 1 | 1 | 1 | 1 | 1 | 1 |
| chr2 | 214437986 | 1 | 1 | 1 | 1 | 1 | 1 |
| chr2 | 221278206 | 1 | 1 | 1 | | 1 | 1 |
| chr2 | 230305997 | 1 | 1 | | | | 1 |
| chr2 | 230341537 | 1 | 1 | 1 | 1 | 1 | 1 |
| chr2 | 231207228 | 1 | 1 | 1 | 1 | 1 | 1 |
| chr2 | 233019939 | 1 | 1 | 1 | 1 | 1 | 1 |
| chr3 | 4004761 | 1 | 1 | 1 | 1 | 1 | 1 |
| chr3 | 4958223 | 1 | 1 | 1 | 1 | 1 | 1 |
| chr3 | 6752704 | | | 1 | | | 1 |
| chr3 | 18134199 | | | | | | |
| chr3 | 19005321 | 1 | 1 | 1 | 1 | 1 | 1 |
| chr3 | 19881024 | | 1 | 1 | | 1 | |
| chr3 | 19896236 | 1 | 1 | 1 | 1 | 1 | 1 |
| chr3 | 20090525 | 1 | | | | 1 | 1 |
| chr3 | 20748859 | 1 | 1 | 1 | 1 | 1 | 1 |
| chr3 | 23106622 | | | | | 1 | 1 |
| chr3 | 26445536 | | | | | | |
| chr3 | 27529306 | 1 | 1 | 1 | 1 | 1 | 1 |
| chr3 | 30419354 | 1 | 1 | 1 | | 1 | 1 |
| chr3 | 36719189 | | | 1 | | | 1 |
| chr3 | 36830677 | 1 | 1 | | | 1 | 1 |
| chr3 | 38626054 | 1 | 1 | 1 | | 1 | 1 |
| chr3 | 43752694 | | | | | | |
| chr3 | 46830625 | 1 | 1 | 1 | 1 | 1 | 1 |
| *chr3 | 53405353 | 1 | 1 | 1 | 1 | 1 | 1 |
| chr3 | 54434350 | 1 | 1 | 1 | 1 | 1 | 1 |
| chr3 | 55788556 | 1 | 1 | 1 | 1 | 1 | 1 |
| chr3 | 56063294 | | | 1 | | | 1 |
| chr3 | 65494968 | 1 | 1 | 1 | 1 | 1 | 1 |
| chr3 | 67735128 | | | | 1 | | 1 |
| chr3 | 75108352 | | | | | | 1 |
| chr3 | 75747827 | | | | | | 1 |
| chr3 | 77818829 | 1 | 1 | 1 | 1 | 1 | 1 |
| chr3 | 79216288 | 1 | 1 | 1 | | 1 | 1 |
| chr3 | 79605276 | | | | | | 1 |
| chr3 | 80590146 | 1 | 1 | 1 | 1 | 1 | 1 |
| chr3 | 81990979 | | 1 | 1 | | 1 | 1 |
| chr3 | 82144843 | 1 | 1 | 1 | 1 | 1 | 1 |
| chr3 | 84433383 | 1 | 1 | 1 | 1 | 1 | 1 |
| chr3 | 85576539 | 1 | 1 | 1 | 1 | 1 | 1 |
| chr3 | 89516006 | 1 | 1 | 1 | 1 | 1 | 1 |
| chr3 | 90224745 | 1 | 1 | 1 | 1 | 1 | 1 |
| chr3 | 90383232 | 1 | 1 | 1 | 1 | 1 | 1 |
| chr3 | 94203237 | | | | | | 1 |
| chr3 | 98775471 | | 1 | 1 | | | 1 |
| chr3 | 101279624 | 1 | 1 | 1 | | 1 | 1 |
| chr3 | 103281410 | 1 | 1 | 1 | 1 | 1 | 1 |
| chr3 | 108468248 | 1 | 1 | 1 | 1 | 1 | 1 |
| chr3 | 108918720 | 1 | 1 | 1 | 1 | 1 | 1 |
| chr3 | 115448700 | | | | | | 1 |
| chr3 | 115834511 | 1 | 1 | 1 | | 1 | 1 |
| chr3 | 120291872 | 1 | 1 | 1 | 1 | 1 | 1 |
| chr3 | 123590700 | 1 | 1 | 1 | 1 | 1 | 1 |
| chr3 | 130861312 | | 1 | | | 1 | 1 |
| chr3 | 131069758 | | 1 | 1 | | 1 | 1 |
| chr3 | 132670878 | 1 | 1 | 1 | 1 | 1 | 1 |
| chr3 | 136203943 | 1 | 1 | 1 | 1 | 1 | 1 |
| chr3 | 136688538 | 1 | 1 | 1 | 1 | 1 | 1 |
| chr3 | 139044403 | 1 | 1 | 1 | | | 1 |
| chr3 | 139135266 | | | | | | 1 |
| chr3 | 139309801 | 1 | 1 | 1 | 1 | 1 | 1 |
| chr3 | 143121747 | 1 | 1 | 1 | | 1 | 1 |
| chr3 | 145663473 | | | | | | 1 |
| chr3 | 151148531 | 1 | 1 | 1 | | 1 | 1 |
| chr3 | 151688069 | 1 | 1 | 1 | | 1 | 1 |
| chr3 | 152701317 | 1 | 1 | 1 | | 1 | 1 |
| chr3 | 157737466 | 1 | 1 | 1 | 1 | 1 | 1 |
| chr3 | 158819183 | 1 | 1 | 1 | 1 | 1 | 1 |
| chr3 | 161048062 | | | | | | 1 |
| chr3 | 162954730 | 1 | 1 | 1 | 1 | 1 | 1 |
| chr3 | 169269939 | 1 | 1 | 1 | 1 | 1 | 1 |
| chr3 | 172749115 | | | 1 | | 1 | 1 |
| chr3 | 186372113 | 1 | 1 | 1 | 1 | 1 | 1 |
| chr3 | 187835296 | | | | | | 1 |
| chr4 | 10632661 | 1 | 1 | 1 | | 1 | 1 |
| chr4 | 13595798 | | | | 1 | | 1 |
| chr4 | 14756739 | 1 | 1 | 1 | 1 | 1 | 1 |
| chr4 | 15843191 | 1 | 1 | 1 | 1 | 1 | 1 |
| chr4 | 15872154 | 1 | 1 | | 1 | 1 | 1 |
| chr4 | 18162708 | | | | | | 1 |
| chr4 | 19085552 | 1 | 1 | 1 | | 1 | 1 |
| chr4 | 21160986 | 1 | 1 | 1 | 1 | 1 | 1 |
| chr4 | 23616398 | 1 | 1 | 1 | 1 | 1 | 1 |
| chr4 | 29894698 | | | | | | 1 |
| chr4 | 35874513 | | | | | | 1 |
| chr4 | 47007769 | | | | | | 1 |
| chr4 | 48059991 | 1 | 1 | 1 | 1 | 1 | 1 |
| chr4 | 53404638 | 1 | 1 | 1 | 1 | 1 | 1 |
| chr4 | 53682630 | 1 | 1 | 1 | 1 | 1 | 1 |
| chr4 | 58434510 | 1 | 1 | 1 | 1 | 1 | 1 |
| chr4 | 59944559 | 1 | 1 | 1 | 1 | 1 | 1 |
| chr4 | 62811677 | 1 | 1 | 1 | 1 | 1 | 1 |
| chr4 | 63295992 | | | | | | 1 |
| chr4 | 63598687 | 1 | 1 | 1 | 1 | 1 | 1 |
| chr4 | 69572737 | 1 | 1 | 1 | | 1 | 1 |
| chr4 | 69653281 | | | | | | 1 |
| chr4 | 74488889 | | | 1 | | | |
| chr4 | 75648794 | 1 | 1 | 1 | 1 | 1 | 1 |
| chr4 | 79026892 | 1 | 1 | 1 | 1 | 1 | 1 |
| chr4 | 79269120 | 1 | 1 | 1 | 1 | 1 | 1 |
| chr4 | 80625707 | 1 | 1 | 1 | 1 | 1 | 1 |
| chr4 | 80858872 | 1 | 1 | 1 | 1 | 1 | 1 |
| chr4 | 80888062 | 1 | 1 | 1 | 1 | 1 | 1 |
| chr4 | 82206549 | | | | | | |
| chr4 | 82921862 | | | 1 | | 1 | |
| chr4 | 84651222 | | | 1 | | | 1 |
| chr4 | 88274300 | 1 | 1 | 1 | 1 | 1 | 1 |
| chr4 | 91602908 | 1 | 1 | 1 | 1 | 1 | 1 |
| chr4 | 91602908 | 1 | 1 | 1 | 1 | 1 | 1 |
| chr4 | 92358649 | 1 | 1 | 1 | 1 | 1 | 1 |
| chr4 | 92899365 | 1 | 1 | 1 | 1 | 1 | 1 |
| chr4 | 94535489 | 1 | 1 | 1 | 1 | 1 | 1 |
| chr4 | 94565490 | 1 | 1 | 1 | 1 | 1 | 1 |
| chr4 | 95906372 | | | | | | 1 |
| chr4 | 98103810 | | | 1 | | 1 | 1 |
| chr4 | 98127347 | | | 1 | | | |
| chr4 | 99519612 | 1 | 1 | 1 | 1 | 1 | 1 |
| chr4 | 107094982 | | | | | | 1 |
| chr4 | 107498227 | 1 | 1 | 1 | 1 | 1 | 1 |
| chr4 | 110248303 | 1 | 1 | 1 | | 1 | 1 |
| chr4 | 112628948 | 1 | 1 | 1 | | 1 | 1 |
| chr4 | 115132690 | | | | | | 1 |
| chr4 | 116660137 | 1 | | | | | 1 |
| chr4 | 120875910 | 1 | 1 | 1 | 1 | 1 | 1 |
| chr4 | 122918574 | | | | | | 1 |
| chr4 | 128965415 | | | | | | 1 |
| chr4 | 132181636 | 1 | 1 | | | 1 | 1 |
| chr4 | 133865549 | 1 | 1 | 1 | 1 | 1 | 1 |
| chr4 | 136104902 | 1 | 1 | 1 | 1 | 1 | 1 |
| chr4 | 137220701 | 1 | 1 | 1 | 1 | 1 | 1 |
| chr4 | 144020626 | 1 | 1 | 1 | 1 | 1 | 1 |
| chr4 | 145757884 | 1 | 1 | 1 | 1 | 1 | 1 |
| chr4 | 147141625 | | | | | | 1 |
| chr4 | 147225281 | 1 | 1 | 1 | 1 | 1 | 1 |
| chr4 | 150798801 | | | | | 1 | 1 |
| chr4 | 152732795 | 1 | 1 | 1 | 1 | 1 | 1 |
| chr4 | 158907262 | 1 | 1 | 1 | | 1 | 1 |
| chr4 | 161691145 | | | 1 | | | 1 |
| chr4 | 167491132 | 1 | 1 | 1 | 1 | 1 | 1 |
| chr4 | 167677051 | 1 | 1 | 1 | 1 | 1 | 1 |
| chr4 | 169413066 | 1 | 1 | 1 | | 1 | 1 |
| chr4 | 190058240 | 1 | 1 | 1 | | 1 | 1 |
| chr5 | 10887243 | | | | | | |
| chr5 | 13231874 | | | | | 1 | 1 |
| chr5 | 13416613 | 1 | 1 | 1 | 1 | 1 | 1 |
| chr5 | 14079084 | | | | | | |
| chr5 | 15912659 | 1 | 1 | 1 | 1 | 1 | 1 |
| chr5 | 15913061 | 1 | 1 | 1 | 1 | 1 | 1 |
| chr5 | 16464296 | 1 | 1 | 1 | | | 1 |
| chr5 | 16882996 | | 1 | 1 | | | 1 |
| chr5 | 21207713 | 1 | 1 | 1 | 1 | 1 | 1 |
| chr5 | 21899840 | 1 | 1 | 1 | | 1 | 1 |
| chr5 | 25708665 | | 1 | | | | 1 |
| chr5 | 30982893 | | | | | | 1 |
| chr5 | 33797529 | 1 | 1 | 1 | | 1 | 1 |
| chr5 | 34147951 | 1 | 1 | 1 | 1 | 1 | 1 |
| chr5 | 35815141 | | | | | | 1 |
| chr5 | 38599852 | | | | | | 1 |
| *chr5 | 39787755 | 1 | 1 | 1 | 1 | 1 | 1 |
| chr5 | 40041321 | 1 | 1 | 1 | | 1 | 1 |
| chr5 | 45605628 | | | | 1 | | 1 |
| chr5 | 53630865 | | | | | | 1 |
| chr5 | 55674629 | | | | | | 1 |
| chr5 | 57680005 | 1 | 1 | 1 | | 1 | 1 |
| chr5 | 68184446 | | | | | | 1 |
| chr5 | 76426440 | | | | | | 1 |
| chr5 | 79080761 | 1 | 1 | 1 | 1 | 1 | 1 |
| chr5 | 80911910 | 1 | 1 | 1 | 1 | 1 | 1 |
| chr5 | 85806511 | 1 | 1 | 1 | 1 | 1 | 1 |
| chr5 | 89450757 | 1 | 1 | 1 | 1 | 1 | 1 |
| chr5 | 89939144 | | | | | | 1 |
| chr5 | 96866406 | | | | | | 1 |
| chr5 | 97932731 | | | | | | 1 |
| chr5 | 101467063 | 1 | 1 | 1 | 1 | 1 | 1 |
| chr5 | 101753808 | | | 1 | | | 1 |
| chr5 | 103854274 | 1 | 1 | 1 | 1 | 1 | 1 |
| chr5 | 104854976 | 1 | 1 | 1 | 1 | 1 | 1 |
| chr5 | 105935350 | 1 | 1 | 1 | | 1 | 1 |
| chr5 | 108595074 | 1 | 1 | 1 | 1 | 1 | 1 |
| chr5 | 109480243 | 1 | 1 | 1 | 1 | 1 | 1 |
| chr5 | 110637937 | 1 | 1 | 1 | 1 | 1 | 1 |
| chr5 | 112703050 | 1 | | 1 | | 1 | 1 |
| chr5 | 115449067 | 1 | 1 | 1 | 1 | 1 | 1 |
| chr5 | 116758570 | | | | | 1 | |
| chr5 | 118935027 | 1 | 1 | 1 | 1 | 1 | 1 |
| chr5 | 119026509 | 1 | 1 | 1 | 1 | 1 | 1 |
| chr5 | 132918964 | 1 | 1 | 1 | 1 | 1 | 1 |
| chr5 | 134707548 | | | | | | 1 |
| chr5 | 137014764 | 1 | 1 | 1 | | 1 | 1 |
| chr5 | 143413864 | | | | | | 1 |
| chr5 | 145989049 | 1 | 1 | 1 | 1 | 1 | 1 |
| chr5 | 151456413 | 1 | 1 | 1 | | 1 | 1 |
| chr5 | 152272033 | 1 | 1 | 1 | 1 | 1 | 1 |
| chr5 | 152456568 | 1 | 1 | 1 | 1 | 1 | 1 |
| chr5 | 155494981 | 1 | 1 | 1 | 1 | 1 | 1 |
| chr5 | 159290926 | | | 1 | | | 1 |
| chr5 | 160142646 | 1 | 1 | 1 | 1 | 1 | 1 |
| chr5 | 164495144 | 1 | 1 | 1 | | 1 | 1 |
| chr5 | 166399820 | 1 | 1 | 1 | 1 | 1 | 1 |
| chr5 | 172835828 | 1 | 1 | 1 | 1 | 1 | 1 |
| chr5 | 177199247 | 1 | 1 | 1 | 1 | 1 | 1 |
| chr6 | 2424034 | 1 | 1 | 1 | 1 | 1 | 1 |
| chr6 | 5558280 | | 1 | | | 1 | 1 |
| chr6 | 13191006 | 1 | 1 | 1 | | | 1 |
| chr6 | 13503024 | 1 | 1 | 1 | 1 | 1 | 1 |
| chr6 | 19765106 | 1 | 1 | 1 | 1 | 1 | 1 |
| chr6 | 19793122 | 1 | | | | | 1 |
| chr6 | 24817950 | 1 | 1 | 1 | | 1 | 1 |
| chr6 | 27956030 | | | 1 | | | 1 |
| chr6 | 29920223 | 1 | 1 | | | | 1 |
| chr6 | 32457266 | | | | | | |
| chr6 | 32589537 | 1 | 1 | 1 | | 1 | 1 |
| chr6 | 32613447 | | | | | | |
| chr6 | 45325822 | | | | | | 1 |
| chr6 | 45934047 | | | | 1 | | 1 |
| *chr6 | 51536770 | 1 | 1 | 1 | 1 | 1 | 1 |
| chr6 | 51739582 | 1 | 1 | 1 | 1 | 1 | 1 |
| chr6 | 63368184 | 1 | 1 | 1 | | 1 | 1 |
| chr6 | 63415756 | | | 1 | | | 1 |
| chr6 | 70720219 | 1 | 1 | 1 | 1 | 1 | 1 |
| chr6 | 72799499 | 1 | 1 | 1 | | 1 | 1 |
| chr6 | 73704406 | 1 | 1 | 1 | 1 | 1 | 1 |
| chr6 | 74649915 | | | | | 1 | 1 |
| chr6 | 77980621 | | 1 | | | | 1 |
| chr6 | 78889134 | | | 1 | | 1 | 1 |
| chr6 | 84043672 | 1 | 1 | 1 | 1 | 1 | 1 |
| chr6 | 85318155 | 1 | 1 | 1 | 1 | 1 | 1 |
| chr6 | 86714791 | 1 | 1 | 1 | 1 | 1 | 1 |
| chr6 | 90952996 | | | | | 1 | |
| chr6 | 94581496 | 1 | 1 | 1 | 1 | 1 | 1 |
| chr6 | 94913772 | 1 | 1 | 1 | 1 | 1 | 1 |
| chr6 | 99150936 | 1 | 1 | 1 | 1 | 1 | 1 |
| chr6 | 100042078 | | | | 1 | | 1 |
| chr6 | 102846070 | 1 | 1 | 1 | | 1 | 1 |
| chr6 | 104013070 | | | | | | 1 |
| chr6 | 108982608 | | | | | | |
| chr6 | 113024951 | 1 | 1 | 1 | 1 | 1 | 1 |
| chr6 | 117395491 | | 1 | | | 1 | 1 |
| chr6 | 117423298 | 1 | 1 | 1 | 1 | 1 | 1 |
| chr6 | 121483863 | 1 | 1 | 1 | 1 | 1 | 1 |
| chr6 | 123853924 | 1 | 1 | 1 | 1 | 1 | 1 |
| chr6 | 125692036 | 1 | | | | | 1 |
| chr6 | 126994697 | 1 | 1 | 1 | 1 | 1 | 1 |
| chr6 | 129319515 | 1 | 1 | 1 | 1 | 1 | 1 |
| chr6 | 133347885 | 1 | 1 | 1 | 1 | 1 | 1 |
| chr6 | 133484973 | | | | | | 1 |
| chr6 | 140318268 | | | | | | 1 |
| chr6 | 142450086 | 1 | 1 | 1 | 1 | 1 | 1 |
| chr6 | 148163559 | 1 | 1 | 1 | | 1 | 1 |
| chr6 | 155647029 | | 1 | | | | 1 |
| chr6 | 155950297 | | | | | | 1 |
| chr6 | 156355272 | 1 | 1 | 1 | 1 | 1 | 1 |
| chr6 | 156652141 | 1 | 1 | 1 | 1 | 1 | 1 |
| chr6 | 157968715 | 1 | 1 | 1 | 1 | 1 | 1 |
| chr6 | 157969109 | 1 | 1 | 1 | 1 | 1 | 1 |
| chr6 | 162181262 | | | | | | 1 |
| chr6 | 162289285 | | | | | | 1 |
| chr7 | 3891219 | 1 | 1 | 1 | 1 | 1 | |
| chr7 | 7504724 | 1 | 1 | 1 | 1 | 1 | 1 |
| chr7 | 8019000 | 1 | 1 | 1 | | 1 | 1 |
| chr7 | 8886704 | 1 | 1 | 1 | | 1 | 1 |
| chr7 | 16661318 | | | | | | 1 |
| chr7 | 21927847 | | | | | | 1 |
| chr7 | 25087510 | 1 | 1 | 1 | 1 | 1 | 1 |
| chr7 | 28009674 | | | | | | 1 |
| chr7 | 30478848 | 1 | 1 | 1 | 1 | 1 | 1 |
| chr7 | 30814160 | | | | | 1 | 1 |
| chr7 | 42529852 | 1 | 1 | 1 | | 1 | 1 |
| chr7 | 43227144 | | | | | | 1 |
| chr7 | 45531876 | | 1 | | | 1 | 1 |
| chr7 | 46501502 | | | | | | 1 |
| chr7 | 47864838 | | | 1 | | | 1 |
| chr7 | 49725896 | 1 | 1 | 1 | 1 | 1 | 1 |
| chr7 | 53652488 | 1 | 1 | 1 | 1 | 1 | 1 |
| chr7 | 54777696 | | | | | 1 | 1 |
| chr7 | 55383721 | | | | | | 1 |
| chr7 | 56652479 | 1 | 1 | 1 | 1 | 1 | 1 |
| chr7 | 61090051 | | | | | | 1 |
| chr7 | 62615237 | 1 | 1 | 1 | 1 | 1 | 1 |
| chr7 | 65757868 | 1 | 1 | 1 | 1 | 1 | 1 |
| chr7 | 66591159 | 1 | 1 | 1 | 1 | 1 | 1 |
| chr7 | 69662318 | 1 | 1 | 1 | 1 | 1 | 1 |
| chr7 | 76723953 | | | | | | |
| chr7 | 86719193 | | | | | | 1 |
| chr7 | 89944383 | 1 | 1 | | | | |
| chr7 | 90331895 | | 1 | | | 1 | 1 |
| chr7 | 90802759 | 1 | 1 | 1 | 1 | 1 | 1 |
| chr7 | 92857551 | | | | | | 1 |
| chr7 | 93422988 | 1 | 1 | 1 | 1 | 1 | 1 |
| chr7 | 96481992 | 1 | 1 | 1 | 1 | 1 | 1 |
| chr7 | 97242972 | 1 | 1 | 1 | 1 | 1 | 1 |
| chr7 | 104319003 | | | | | 1 | 1 |
| chr7 | 105967850 | | | 1 | | | 1 |
| chr7 | 106398050 | | | | | | 1 |
| chr7 | 107829239 | 1 | 1 | 1 | 1 | 1 | 1 |
| chr7 | 110353080 | 1 | 1 | 1 | 1 | 1 | 1 |
| chr7 | 110889602 | 1 | 1 | 1 | 1 | 1 | 1 |
| chr7 | 111603252 | 1 | 1 | 1 | 1 | 1 | 1 |
| chr7 | 113422207 | 1 | 1 | 1 | 1 | 1 | 1 |
| chr7 | 116270339 | | | | | | 1 |
| chr7 | 125291681 | | | | | | 1 |
| chr7 | 130867411 | | | | | | 1 |
| chr7 | 134844920 | | | | | | 1 |
| chr7 | 140767839 | 1 | 1 | 1 | 1 | 1 | 1 |
| chr7 | 141626512 | 1 | 1 | 1 | 1 | 1 | 1 |
| chr7 | 142818636 | | 1 | 1 | | 1 | 1 |
| chr7 | 146795524 | | | | | | |
| chr7 | 147545712 | 1 | 1 | 1 | 1 | 1 | 1 |
| chr7 | 149909484 | | 1 | 1 | | | 1 |
| chr8 | 8328372 | 1 | 1 | 1 | 1 | 1 | 1 |
| chr8 | 9469163 | 1 | 1 | 1 | 1 | 1 | 1 |
| chr8 | 11164185 | | | | 1 | | 1 |
| chr8 | 17190306 | | | | | | 1 |
| chr8 | 26971136 | | 1 | | | | 1 |
| chr8 | 33625997 | 1 | 1 | 1 | 1 | 1 | 1 |
| chr8 | 40433163 | | | | | | 1 |
| chr8 | 57161899 | 1 | 1 | 1 | | 1 | 1 |
| chr8 | 72399210 | 1 | | 1 | | 1 | 1 |
| chr8 | 72399225 | 1 | 1 | 1 | 1 | 1 | 1 |
| chr8 | 73793823 | 1 | 1 | 1 | 1 | 1 | 1 |
| chr8 | 75211408 | 1 | 1 | | | | |
| chr8 | 76539590 | 1 | 1 | 1 | 1 | 1 | 1 |
| chr8 | 84573946 | 1 | 1 | | | 1 | 1 |
| chr8 | 85428593 | 1 | 1 | 1 | 1 | 1 | 1 |
| chr8 | 89697938 | 1 | 1 | 1 | 1 | 1 | 1 |
| chr8 | 92540349 | | | | | | |
| chr8 | 100525412 | | | | | | 1 |
| chr8 | 105758098 | 1 | 1 | 1 | 1 | 1 | 1 |
| chr8 | 105977058 | 1 | 1 | 1 | 1 | 1 | 1 |
| chr8 | 109052010 | | | | | | |
| chr8 | 109649024 | | | 1 | | | 1 |
| chr8 | 123643859 | | | 1 | | | 1 |
| chr8 | 126595121 | 1 | 1 | 1 | 1 | 1 | 1 |
| chr8 | 129471266 | 1 | 1 | 1 | 1 | 1 | 1 |
| chr8 | 131041219 | | | | | | 1 |
| chr8 | 134889233 | 1 | 1 | 1 | 1 | | 1 |
| chr8 | 135089016 | 1 | 1 | 1 | 1 | 1 | 1 |
| chr8 | 136888106 | 1 | 1 | 1 | 1 | 1 | 1 |
| chr8 | 137223447 | 1 | | | | | 1 |
| chr8 | 137456345 | 1 | 1 | 1 | 1 | 1 | 1 |
| chr8 | 140145234 | | | 1 | 1 | | 1 |
| chr9 | 1640113 | | | | | | 1 |
| chr9 | 5491391 | 1 | 1 | 1 | 1 | 1 | 1 |
| chr9 | 5491404 | 1 | 1 | 1 | 1 | 1 | 1 |
| chr9 | 7741384 | 1 | 1 | 1 | 1 | 1 | 1 |
| chr9 | 18584286 | | | | | | 1 |
| chr9 | 28802507 | | | 1 | | | 1 |
| chr9 | 32757316 | | | | | | 1 |
| chr9 | 46445703 | | | | | | |
| chr9 | 72318334 | 1 | | 1 | | | 1 |
| chr9 | 72777388 | 1 | 1 | 1 | | 1 | 1 |
| chr9 | 73521475 | | | | | | 1 |
| chr9 | 74904890 | 1 | 1 | 1 | | 1 | 1 |
| chr9 | 85670507 | 1 | 1 | 1 | 1 | 1 | 1 |
| chr9 | 89891668 | 1 | 1 | | | 1 | 1 |
| chr9 | 91774009 | | | 1 | | | 1 |
| chr9 | 94416755 | 1 | 1 | 1 | 1 | 1 | 1 |
| chr9 | 96881847 | 1 | 1 | 1 | 1 | 1 | 1 |
| chr9 | 98459868 | 1 | 1 | 1 | 1 | 1 | 1 |
| chr9 | 99364841 | | | | | | 1 |
| chr9 | 103870458 | 1 | 1 | 1 | 1 | 1 | 1 |
| chr9 | 107636009 | | | | | | 1 |
| chr9 | 111564944 | 1 | 1 | 1 | 1 | 1 | 1 |
| chr9 | 113179386 | 1 | 1 | 1 | 1 | 1 | 1 |
| chr9 | 113553378 | 1 | 1 | 1 | 1 | 1 | 1 |
| chr9 | 115566439 | 1 | 1 | 1 | 1 | 1 | 1 |
| chr9 | 121394567 | | | 1 | | | 1 |
| chr10 | 1427634 | | | 1 | | | 1 |
| chr10 | 4137263 | 1 | 1 | 1 | 1 | 1 | 1 |
| chr10 | 5287299 | 1 | 1 | 1 | 1 | 1 | 1 |
| chr10 | 6417629 | | | | | 1 | 1 |
| chr10 | 8024988 | 1 | 1 | 1 | | 1 | 1 |
| chr10 | 9525633 | | | 1 | | | 1 |
| chr10 | 12969532 | 1 | | | | 1 | 1 |
| chr10 | 14310833 | | | | | | 1 |
| chr10 | 15644911 | | | | | | 1 |
| chr10 | 17310898 | 1 | 1 | 1 | 1 | 1 | 1 |
| chr10 | 19065785 | 1 | 1 | 1 | 1 | 1 | 1 |
| chr10 | 19383547 | | | 1 | | | 1 |
| chr10 | 25576907 | 1 | 1 | 1 | 1 | 1 | 1 |
| chr10 | 25707676 | 1 | 1 | | | 1 | 1 |
| chr10 | 31517470 | | | 1 | | | |
| chr10 | 33315318 | | | | | | 1 |
| chr10 | 33805804 | 1 | 1 | 1 | 1 | 1 | 1 |
| chr10 | 36759271 | 1 | | | | | 1 |
| chr10 | 60914465 | | | 1 | 1 | | 1 |
| chr10 | 61148598 | | | | | | 1 |
| chr10 | 68949307 | 1 | 1 | 1 | | 1 | 1 |
| chr10 | 86297452 | | | | | | 1 |
| chr10 | 87115266 | 1 | 1 | 1 | 1 | 1 | 1 |
| chr10 | 91717619 | 1 | 1 | 1 | 1 | 1 | 1 |
| chr10 | 94292473 | | | | 1 | 1 | 1 |
| chr10 | 100548725 | 1 | 1 | 1 | 1 | 1 | 1 |
| chr10 | 107143135 | 1 | 1 | 1 | 1 | 1 | 1 |
| chr10 | 107541306 | 1 | 1 | 1 | 1 | 1 | 1 |
| chr10 | 110075897 | 1 | 1 | 1 | 1 | 1 | 1 |
| chr10 | 111578216 | 1 | 1 | 1 | | 1 | 1 |
| chr10 | 122662164 | | 1 | 1 | | | 1 |
| chr10 | 124455206 | 1 | 1 | 1 | | 1 | 1 |
| chr10 | 126983613 | | | 1 | | 1 | 1 |
| chr10 | 130625027 | 1 | 1 | 1 | 1 | 1 | 1 |
| chr10 | 131468370 | | 1 | | | | 1 |
| chr11 | 5247056 | | | 1 | | | 1 |
| chr11 | 10042418 | | 1 | 1 | | | 1 |
| chr11 | 13944123 | | | 1 | | | |
| chr11 | 14647682 | | | | | | 1 |
| *chr11 | 14737455 | 1 | 1 | 1 | 1 | 1 | 1 |
| chr11 | 19909956 | | | | 1 | | 1 |
| chr11 | 24349498 | 1 | 1 | 1 | 1 | 1 | 1 |
| chr11 | 30180441 | | | 1 | | 1 | 1 |
| chr11 | 31343227 | 1 | 1 | 1 | 1 | 1 | 1 |
| chr11 | 34802464 | | | | | | 1 |
| chr11 | 34966598 | | | | | | 1 |
| chr11 | 36558481 | 1 | 1 | 1 | | 1 | 1 |
| chr11 | 36579184 | 1 | 1 | 1 | 1 | 1 | 1 |
| chr11 | 45157045 | | | | | | 1 |
| chr11 | 48367510 | 1 | 1 | 1 | 1 | 1 | 1 |
| chr11 | 48875276 | 1 | 1 | 1 | 1 | 1 | 1 |
| chr11 | 55870743 | 1 | 1 | 1 | | 1 | 1 |
| chr11 | 57985411 | 1 | | | | 1 | 1 |
| chr11 | 58512603 | | | | | | |
| chr11 | 63048427 | | | | | | 1 |
| chr11 | 67599050 | 1 | 1 | 1 | 1 | 1 | 1 |
| chr11 | 72808550 | | | | | | 1 |
| chr11 | 73696777 | 1 | | | 1 | | |
| chr11 | 78394847 | 1 | 1 | 1 | 1 | 1 | 1 |
| chr11 | 81866908 | 1 | 1 | 1 | 1 | 1 | 1 |
| chr11 | 83313943 | 1 | 1 | | | 1 | 1 |
| chr11 | 83652337 | 1 | 1 | | 1 | 1 | 1 |
| chr11 | 85035805 | 1 | 1 | 1 | 1 | 1 | 1 |
| chr11 | 87057100 | 1 | 1 | 1 | 1 | 1 | 1 |
| chr11 | 87566794 | 1 | | | | | 1 |
| chr11 | 90139264 | 1 | 1 | 1 | 1 | 1 | 1 |
| chr11 | 90705467 | 1 | 1 | 1 | 1 | 1 | 1 |
| chr11 | 92869786 | 1 | 1 | 1 | | 1 | 1 |
| chr11 | 93154153 | 1 | 1 | 1 | | 1 | 1 |
| chr11 | 95169369 | 1 | 1 | 1 | 1 | 1 | 1 |
| chr11 | 100712182 | | | | | | 1 |
| chr11 | 109054251 | 1 | 1 | 1 | 1 | 1 | 1 |
| chr11 | 110377868 | 1 | 1 | 1 | 1 | 1 | 1 |
| chr11 | 114895494 | 1 | 1 | 1 | | 1 | 1 |
| chr11 | 117195201 | | 1 | | | | 1 |
| chr11 | 119327942 | | | 1 | | 1 | 1 |
| chr11 | 125406506 | 1 | 1 | 1 | 1 | 1 | 1 |
| chr11 | 128141113 | | 1 | | | | 1 |
| chr11 | 132682319 | | | | | | 1 |
| chr12 | 3614391 | 1 | 1 | 1 | 1 | 1 | 1 |
| chr12 | 12933683 | | | 1 | | | |
| chr12 | 24868692 | 1 | 1 | | | 1 | 1 |
| chr12 | 25775838 | 1 | 1 | 1 | | 1 | 1 |
| chr12 | 28226400 | 1 | 1 | 1 | 1 | 1 | 1 |
| chr12 | 31353750 | 1 | 1 | 1 | 1 | 1 | 1 |
| chr12 | 33325774 | 1 | 1 | 1 | | 1 | 1 |
| chr12 | 34017331 | 1 | 1 | 1 | 1 | 1 | 1 |
| chr12 | 34397661 | | | 1 | | | 1 |
| chr12 | 39004810 | | | | | | 1 |
| chr12 | 39193449 | 1 | 1 | 1 | 1 | 1 | 1 |
| chr12 | 43125233 | | | 1 | | | 1 |
| chr12 | 44508034 | 1 | 1 | 1 | 1 | 1 | 1 |
| chr12 | 51962438 | 1 | 1 | 1 | 1 | 1 | 1 |
| chr12 | 53169783 | | | 1 | | | 1 |
| chr12 | 55182361 | 1 | 1 | 1 | 1 | 1 | 1 |
| chr12 | 55496064 | 1 | 1 | 1 | 1 | 1 | 1 |
| chr12 | 55685353 | | | | | | 1 |
| chr12 | 55782210 | | 1 | 1 | | | 1 |
| chr12 | 58583122 | | | | | | 1 |
| chr12 | 65689256 | | | | | | 1 |
| chr12 | 71020015 | 1 | 1 | 1 | | 1 | 1 |
| chr12 | 71053959 | 1 | 1 | 1 | 1 | 1 | 1 |
| chr12 | 73683448 | 1 | 1 | 1 | 1 | 1 | 1 |
| chr12 | 75268652 | 1 | 1 | 1 | 1 | 1 | 1 |
| chr12 | 92326094 | | | | | | 1 |
| chr12 | 96912569 | 1 | 1 | | 1 | | 1 |
| chr12 | 97365515 | | | 1 | | 1 | 1 |
| chr12 | 101539822 | 1 | 1 | 1 | 1 | 1 | 1 |
| chr12 | 117814427 | 1 | 1 | 1 | 1 | 1 | 1 |
| chr12 | 126789597 | 1 | 1 | 1 | 1 | 1 | 1 |
| chr12 | 126802915 | 1 | 1 | 1 | | 1 | 1 |
| chr13 | 19557911 | 1 | 1 | 1 | 1 | 1 | 1 |
| chr13 | 19620965 | | | | 1 | 1 | 1 |
| chr13 | 20262500 | | | | | | 1 |
| chr13 | 27339037 | | | | | | 1 |
| chr13 | 30221843 | 1 | 1 | 1 | 1 | 1 | 1 |
| chr13 | 31876452 | 1 | 1 | 1 | 1 | 1 | 1 |
| chr13 | 33390340 | | | 1 | | | 1 |
| chr13 | 35060428 | 1 | 1 | 1 | 1 | 1 | 1 |
| chr13 | 41006626 | | | | | | 1 |
| chr13 | 49039692 | 1 | 1 | 1 | 1 | 1 | 1 |
| chr13 | 55237995 | | 1 | 1 | | 1 | 1 |
| chr13 | 58900843 | | | | | | 1 |
| chr13 | 61462321 | 1 | 1 | 1 | 1 | 1 | 1 |
| chr13 | 61681769 | 1 | 1 | 1 | | 1 | 1 |
| chr13 | 70521020 | | | 1 | 1 | | 1 |
| chr13 | 74613182 | 1 | 1 | 1 | | 1 | 1 |
| chr13 | 75000464 | 1 | 1 | 1 | 1 | 1 | 1 |
| chr13 | 76280374 | | 1 | | | | 1 |
| chr13 | 76618912 | | | 1 | | | 1 |
| chr13 | 77192984 | 1 | 1 | 1 | 1 | 1 | 1 |
| chr13 | 88475686 | | | | | | 1 |
| chr13 | 89423371 | | | | | | 1 |
| chr13 | 93343843 | 1 | 1 | 1 | 1 | 1 | 1 |
| chr13 | 98325691 | 1 | 1 | 1 | 1 | 1 | 1 |
| chr13 | 99519881 | | | 1 | | | 1 |
| chr13 | 107845335 | | | | | 1 | 1 |
| chr13 | 109168843 | 1 | 1 | 1 | 1 | 1 | 1 |
| chr14 | 19066338 | 1 | 1 | 1 | 1 | 1 | 1 |
| chr14 | 24992895 | 1 | 1 | 1 | 1 | 1 | 1 |
| chr14 | 31150794 | 1 | 1 | 1 | 1 | 1 | 1 |
| chr14 | 31160043 | 1 | 1 | 1 | 1 | 1 | 1 |
| chr14 | 34140114 | 1 | 1 | 1 | | 1 | 1 |
| chr14 | 45180304 | 1 | 1 | 1 | 1 | 1 | 1 |
| chr14 | 45952370 | 1 | 1 | 1 | 1 | 1 | 1 |
| chr14 | 48656289 | 1 | 1 | 1 | 1 | 1 | 1 |
| chr14 | 49621405 | | | | | | 1 |
| chr14 | 52267350 | 1 | 1 | 1 | 1 | 1 | 1 |
| chr14 | 52667762 | 1 | 1 | 1 | 1 | 1 | 1 |
| chr14 | 58500001 | 1 | 1 | 1 | 1 | 1 | 1 |
| chr14 | 59160875 | | | 1 | | 1 | 1 |
| chr14 | 59220376 | 1 | 1 | 1 | 1 | 1 | 1 |
| chr14 | 61527866 | 1 | 1 | 1 | 1 | 1 | 1 |
| chr14 | 63002570 | 1 | 1 | 1 | 1 | 1 | 1 |
| chr14 | 63233742 | | | 1 | | | |
| chr14 | 63589447 | 1 | 1 | 1 | 1 | 1 | 1 |
| chr14 | 67419854 | 1 | 1 | 1 | | 1 | 1 |
| chr14 | 68784217 | | | 1 | | 1 | 1 |
| chr14 | 71014014 | 1 | 1 | 1 | 1 | 1 | 1 |
| chr14 | 71197769 | 1 | 1 | 1 | 1 | 1 | 1 |
| chr14 | 80536681 | | | 1 | | | 1 |
| chr14 | 81477209 | 1 | | 1 | | | 1 |
| chr14 | 86149568 | | | | 1 | | 1 |
| chr14 | 86381784 | 1 | 1 | 1 | 1 | 1 | 1 |
| chr14 | 86952502 | | | 1 | | | |
| chr14 | 94642967 | | | | 1 | | |
| chr15 | 20069064 | | | | | | |
| chr15 | 26673803 | | | 1 | | | 1 |
| chr15 | 33812908 | | | | 1 | | 1 |
| chr15 | 34031826 | 1 | 1 | 1 | 1 | 1 | 1 |
| chr15 | 35654118 | | | 1 | | 1 | 1 |
| chr15 | 46335558 | 1 | 1 | 1 | | | 1 |
| chr15 | 47507319 | 1 | 1 | 1 | 1 | 1 | 1 |
| chr15 | 51715443 | 1 | 1 | 1 | 1 | 1 | 1 |
| chr15 | 51843791 | | | | | | 1 |
| chr15 | 55129512 | | | 1 | | 1 | 1 |
| chr15 | 55224297 | | | 1 | | 1 | |
| chr15 | 56251126 | 1 | 1 | 1 | 1 | 1 | 1 |
| chr15 | 61756146 | | | | | | 1 |
| chr15 | 71021839 | 1 | 1 | 1 | 1 | 1 | 1 |
| chr15 | 80619533 | 1 | 1 | | | 1 | 1 |
| chr15 | 82501484 | | | | | | 1 |
| chr15 | 83551609 | | | | | | |
| chr15 | 83557671 | 1 | 1 | 1 | 1 | 1 | 1 |
| chr15 | 83557685 | 1 | 1 | 1 | 1 | 1 | 1 |
| chr15 | 84125586 | 1 | 1 | 1 | | 1 | 1 |
| chr15 | 85140891 | | | | | | |
| chr15 | 85649314 | 1 | 1 | 1 | 1 | 1 | 1 |
| chr15 | 88053123 | 1 | 1 | 1 | 1 | 1 | 1 |
| chr15 | 89103494 | | | | | | 1 |
| chr15 | 97226910 | | | | | | 1 |
| chr15 | 100432259 | | | | | | 1 |
| chr15 | 101238579 | | | | | | |
| chr16 | 871889 | | | 1 | | 1 | 1 |
| chr16 | 5196792 | 1 | 1 | 1 | 1 | 1 | 1 |
| chr16 | 6119007 | | | | | | 1 |
| chr16 | 9684379 | 1 | 1 | 1 | 1 | 1 | 1 |
| chr16 | 16940414 | 1 | 1 | 1 | | 1 | 1 |
| chr16 | 27425913 | 1 | 1 | 1 | | 1 | 1 |
| chr16 | 32525600 | | | | | | 1 |
| chr16 | 33761079 | 1 | 1 | 1 | 1 | 1 | 1 |
| chr16 | 34848872 | 1 | 1 | 1 | 1 | 1 | 1 |
| chr16 | 54076009 | 1 | 1 | 1 | 1 | 1 | 1 |
| chr16 | 62491608 | | | | | | 1 |
| chr16 | 65723915 | 1 | 1 | 1 | 1 | 1 | 1 |
| chr16 | 68623408 | 1 | 1 | 1 | 1 | 1 | 1 |
| chr16 | 71027998 | | | | | | 1 |
| chr16 | 71294354 | | | | | | 1 |
| chr16 | 72522723 | | | | | | 1 |
| chr16 | 76539036 | | | | | | 1 |
| chr16 | 83670839 | 1 | 1 | 1 | 1 | 1 | 1 |
| chr17 | 9519303 | 1 | 1 | 1 | 1 | 1 | 1 |
| chr17 | 12359254 | 1 | 1 | 1 | 1 | 1 | 1 |
| chr17 | 18776439 | | | | | | 1 |
| chr17 | 29657360 | | | | | | 1 |
| chr17 | 31640351 | | | | | | 1 |
| chr17 | 33135397 | | | 1 | 1 | 1 | 1 |
| chr17 | 61281439 | 1 | 1 | | | | 1 |
| chr17 | 64598710 | 1 | 1 | 1 | 1 | 1 | 1 |
| chr17 | 64637274 | 1 | 1 | 1 | 1 | 1 | 1 |
| chr17 | 68357385 | 1 | 1 | 1 | 1 | 1 | 1 |
| chr17 | 68455086 | 1 | 1 | 1 | 1 | 1 | 1 |
| chr17 | 68546936 | 1 | 1 | 1 | 1 | 1 | 1 |
| chr18 | 535704 | 1 | 1 | 1 | 1 | 1 | 1 |
| chr18 | 12491251 | 1 | 1 | 1 | | 1 | 1 |
| chr18 | 13981887 | 1 | 1 | 1 | 1 | 1 | 1 |
| chr18 | 15095248 | 1 | 1 | 1 | 1 | 1 | 1 |
| chr18 | 35399687 | 1 | 1 | 1 | 1 | 1 | 1 |
| chr18 | 39354983 | 1 | 1 | 1 | 1 | 1 | 1 |
| chr18 | 39896944 | | | 1 | | 1 | |
| chr18 | 45186745 | 1 | 1 | 1 | 1 | 1 | 1 |
| chr18 | 47876356 | 1 | 1 | 1 | 1 | 1 | 1 |
| chr18 | 49216206 | 1 | 1 | 1 | | 1 | 1 |
| chr18 | 51425715 | 1 | 1 | 1 | | 1 | 1 |
| chr18 | 51451044 | | | | | 1 | 1 |
| chr18 | 55880937 | | | | | | 1 |
| *chr18 | 57077203 | 1 | 1 | 1 | 1 | 1 | 1 |
| chr18 | 60573526 | 1 | 1 | 1 | 1 | 1 | 1 |
| chr18 | 68419817 | 1 | 1 | 1 | 1 | 1 | 1 |
| chr18 | 70513902 | | | | | 1 | 1 |
| chr18 | 70639791 | 1 | 1 | 1 | 1 | 1 | 1 |
| chr18 | 71114387 | | | | | 1 | 1 |
| chr18 | 73290618 | 1 | | 1 | | 1 | 1 |
| chr18 | 73558810 | 1 | 1 | 1 | 1 | 1 | 1 |
| chr19 | 29950184 | 1 | 1 | 1 | 1 | 1 | 1 |
| chr19 | 40188337 | | | | | | |
| chr19 | 44979592 | | | 1 | 1 | | 1 |
| chr19 | 47024925 | | 1 | 1 | | 1 | 1 |
| chr20 | 7102846 | 1 | 1 | 1 | 1 | 1 | 1 |
| chr20 | 9477902 | 1 | 1 | 1 | | 1 | 1 |
| chr20 | 11613428 | 1 | 1 | 1 | 1 | 1 | 1 |
| chr20 | 12787692 | 1 | 1 | 1 | 1 | 1 | 1 |
| chr20 | 17860913 | | | 1 | | 1 | 1 |
| chr20 | 20863919 | | | | 1 | | 1 |
| chr20 | 22501620 | | | 1 | | 1 | 1 |
| chr20 | 22959388 | | | | | | 1 |
| chr20 | 23406746 | 1 | 1 | 1 | 1 | 1 | 1 |
| chr20 | 30474324 | | | | 1 | | 1 |
| chr20 | 50967353 | | 1 | | | 1 | 1 |
| chr20 | 54434610 | 1 | 1 | 1 | 1 | 1 | 1 |
| chr20 | 59972432 | 1 | 1 | 1 | 1 | 1 | 1 |
| chr21 | 19082370 | | | | | | 1 |
| chr21 | 23257888 | | | | | 1 | 1 |
| chr21 | 23626197 | 1 | 1 | 1 | 1 | 1 | 1 |
| chr21 | 25309402 | | | 1 | 1 | | 1 |
| chr21 | 32261329 | 1 | | | | 1 | 1 |
| chr22 | 17246055 | 1 | 1 | 1 | | 1 | 1 |
| chr22 | 19210889 | 1 | 1 | | 1 | 1 | 1 |
| chr22 | 22486356 | 1 | 1 | 1 | 1 | 1 | 1 |
| chr22 | 22715563 | 1 | 1 | 1 | 1 | 1 | 1 |
| chr22 | 26507199 | | | | | | 1 |
| chr22 | 28455902 | 1 | 1 | 1 | | 1 | 1 |
| *chr22 | 29059274 | 1 | 1 | 1 | 1 | 1 | 1 |
| chr22 | 34951700 | 1 | 1 | 1 | 1 | 1 | 1 |
| chr22 | 49387604 | 1 | 1 | 1 | 1 | 1 | 1 |
| chrX | 3879089 | | | | | | 1 |
| chrX | 11725352 | 1 | 1 | 1 | 1 | 1 | 1 |
| chrX | 11959435 | 1 | 1 | 1 | 1 | 1 | 1 |
| chrX | 18352718 | | | | | | 1 |
| chrX | 23256636 | 1 | 1 | 1 | 1 | 1 | 1 |
| chrX | 26338563 | 1 | 1 | 1 | 1 | 1 | 1 |
| chrX | 31015886 | | | | | | 1 |
| chrX | 33425638 | 1 | 1 | 1 | | 1 | 1 |
| chrX | 33425670 | 1 | 1 | 1 | | 1 | 1 |
| chrX | 35063807 | | | | | | |
| chrX | 35064107 | | 1 | | | 1 | 1 |
| chrX | 46697270 | | | | | | 1 |
| chrX | 49790114 | 1 | 1 | 1 | 1 | 1 | 1 |
| chrX | 54151177 | 1 | 1 | 1 | 1 | 1 | 1 |
| chrX | 56421831 | 1 | 1 | 1 | 1 | 1 | 1 |
| chrX | 56722321 | 1 | 1 | 1 | 1 | 1 | 1 |
| chrX | 63239163 | 1 | 1 | 1 | 1 | 1 | 1 |
| chrX | 65406567 | 1 | 1 | 1 | 1 | 1 | 1 |
| chrX | 68696852 | 1 | 1 | 1 | | 1 | |
| chrX | 68696870 | 1 | 1 | 1 | | 1 | |
| chrX | 72325985 | 1 | 1 | 1 | 1 | 1 | 1 |
| chrX | 72600829 | 1 | 1 | 1 | 1 | 1 | 1 |
| chrX | 75549215 | 1 | 1 | 1 | 1 | 1 | 1 |
| chrX | 81096640 | 1 | 1 | 1 | 1 | 1 | 1 |
| chrX | 82320643 | 1 | 1 | 1 | 1 | 1 | 1 |
| chrX | 82797405 | 1 | 1 | 1 | 1 | 1 | 1 |
| chrX | 85213943 | | | | | | 1 |
| chrX | 87076411 | 1 | 1 | 1 | 1 | 1 | 1 |
| chrX | 94852497 | | | | | 1 | 1 |
| chrX | 94852538 | | | | | 1 | 1 |
| chrX | 105712516 | 1 | 1 | 1 | 1 | 1 | 1 |
| chrX | 111229543 | 1 | 1 | 1 | | 1 | 1 |
| chrX | 111229571 | 1 | 1 | 1 | | 1 | 1 |
| chrX | 111557736 | 1 | 1 | 1 | | 1 | 1 |
| chrX | 111557758 | 1 | 1 | 1 | | 1 | 1 |
| chrX | 118575456 | 1 | 1 | 1 | 1 | 1 | 1 |
| chrX | 123921234 | | 1 | 1 | | | 1 |
| chrX | 123921260 | | 1 | 1 | | | 1 |
| chrX | 129525269 | | | | | | 1 |
| chrX | 129651352 | 1 | 1 | 1 | 1 | 1 | 1 |
| chrX | 140515246 | 1 | 1 | 1 | 1 | 1 | 1 |
| chrX | 140515272 | 1 | 1 | 1 | 1 | 1 | 1 |
| chrX | 141565636 | 1 | 1 | 1 | 1 | 1 | 1 |
| chrX | 143704658 | | | 1 | | 1 | 1 |
| chrX | 146735256 | 1 | 1 | 1 | 1 | 1 | 1 |
| chrX | 148265715 | 1 | 1 | 1 | 1 | 1 | 1 |
| chrX | 154751709 | 1 | 1 | 1 | 1 | 1 | 1 |
| chrY | 3311594 | 1 | 1 | 1 | | 1 | 1 |
| chrY | 4822979 | 1 | 1 | | | 1 | 1 |
| chrY | 5480237 | 1 | 1 | 1 | 1 | 1 | 1 |
| chrY | 9778740 | 1 | 1 | 1 | | 1 | 1 |
| chrY | 9954640 | | | | | | 1 |

Legend for Table 2:
“Chrom” and “chr”: chromosome.
“WGS”: acronym for “Whole genome sequencing.”
“SCORE”: acronym for “Screen for Content Of Retro Elements.”
“Cont WGS”: genomes from individuals who did not have Alzheimer's Disease (“AD”) and who had not been diagnosed with cancer, as analyzed by whole genome sequencing. The presence of the number “1” in a row corresponding to a particular chromosome and insertion site within that chromosome indicates that a L1 element was found in the genome of at least one person in the data set analyzed who had not been diagnosed with either AD or cancer. For example, in chromomsome Y, position 3311594 is known to be an insertion site at which a pL1 has been found to insert. The number “1” in the “Cont WGS” column in the row listed for chromosome Y, insertion site 3311594, indicates that the analysis of the data set from individuals who had not been diagnosed with either AD or cancer found that the genome of at least one individual in the data set had a pL1 located at that insertion site. For clarity, it is noted that the appearance of the digit “1” in this column, or in the other columns showing whether a pL1 was present at an insertion site, indicates only that at least one genome of a person in the group labeled at the top of the column was found to have a pL1 at the indicated location; it does not indicate that only one pL1 was found at that location among the genomes of persons in the group indicated.
“AD WGS”: genomes from individuals who were diagnosed with Alzheimer's Disease (“AD”), as analyzed by whole genome sequencing. As stated in the preceding paragraph, the number “1” in this column with respect to a row designating a particular L1 element indicates that the L1 element whose insertion point is named in the two left columns was found in the genome of at least one person in the data set used who had been diagnosed with AD, not the number of genomes in that data set which had a pL1 element present at that position..
“BC WGS”: genomes from individuals who were diagnosed with breast cancer, as analyzed by whole genome sequencing. The number “1” in this column with respect to a row designating a particular L1 element indicates that the L1 element whose insertion point is named in the two left columns was found in the genome of at least one person in the data set used who had been diagnosed with breast cancer.
“BC SCORE”: genome(s) from individual(s) who were diagnosed with breast cancer and analyzed by the inventive methods. The number “1” in this column with respect to a row designating a particular L1 element indicates that the L1 element whose insertion point is named in the two left columns was found in the genome of at least one person in the data set used who had been diagnosed with breast cancer.
“PC WGS”: genomes from individuals who were diagnosed with prostate cancer, as analyzed by whole genome sequencing. The number “1” in this column with respect to a row designating a particular L1 element indicates that the L1 element whose insertion point is named in the two left columns was found in the genome of at least one person in the data set used who had been diagnosed with prostate cancer.
“PC SCORE”: genomes from individuals who were diagnosed with prostate cancer and analyzed by the inventive methods. The number “1” in this column with respect to a row designating a particular L1 element indicates that the L1 element whose insertion point is named in the two left columns was found in the genome of at least one person in the data set used who had been diagnosed with prostate cancer.
Chromosome positions marked with an asterisk designate the positions in the genome of fixed L1s that have been found to be active in causing mutations that can lead to cancers. As they are in every human genome, they are not themselves indicative of an increased risk of cancer compared to anyone else in the population.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition for determining how many polymorphic LINE-1 elements (“pL1s”) which pL1s have a 5′ untranslated region (“5′UTR”) and a 3′UTR, which 5′UTR begins with a contiguous sequence of at least 300 bases and which 3′UTR terminates in a contiguous sequence of at least 300 bases, are present in genomic DNA of a subject, and at which of the sites at which pL1s are known to insert said pL1s are present in said genomic DNA of said subject, said composition comprising
(a) a substrate or a plurality of substrates,
(b) a plurality of first DNA probes, RNA probes, or both, attached to said substrate or said plurality of substrates, each of said first DNA probes, RNA probes, or both, comprising a contiguous sequence of about 200 to about 1000 bases complementary to a consensus human genomic sequence surrounding and including one particular known pL1 insertion site, for each of the pL1 insertion points shown on Table 2, and
(c) a plurality of second DNA probes, RNA probes, or both, which second DNA probes, RNA probes, or both, are complementary to said beginning contiguous sequence of said 300 bases of said 5′UTR of said pL1 or to said 3′UTR contiguous sequence of at least 300 bases.

2. The composition of claim 1, further wherein said first DNA probes, RNA probes, or both, comprise a contiguous sequence of about 200 to about 700 bases.

3-5. (canceled)

6. The composition of claim 1, wherein said substrate is a well of a multi-well plate.

7. The composition of claim 1, wherein said substrate is a wall of a microfluidic device.

8. The composition of claim 1, further wherein some or all of said solid substrates is in the form of beads.

9. (canceled)

10. (canceled)

11. The composition of claim 1, further wherein said plurality of solid surfaces is of plastic.

12. The composition of claim 1, further wherein said attachment of said first DNA probes or said second DNA probe, or both, to said solid support or said plurality of solid supports is covalent.

13. The composition of claim 1, further comprising (d) a plurality of third DNA probes, RNA probes, or both, attached to said substrate or said plurality of substrates, each of said third DNA probes, RNA probes, or both, comprising a contiguous sequence of about 200 to about 1000 bases complementary to a consensus human genomic sequence surrounding and including one or more particular fixed L1 insertion points associated with cancer.

14-21. (canceled)

22. A method for determining if an individual has a risk of developing cancer or Alzheimer's Disease due to polymorphic LINE-1 elements (“pL1s”) related to risk of cancer or Alzheimer's Disease in said individual's genome, said method comprising, determining if said individual carries one of more pL1s and, if so, how many, selected from the following groups:
(a) pL1s identified in Table 2 as found by WGS, SCORE, or both, only in individuals diagnosed with breast cancer,
(b) pL1s identified in Table 2 as found by WGS, SCORE, or both, only in individuals diagnosed with prostate cancer,
(c) pL1s identified in Table 2 as found by WGS, SCORE, or both, in genomes of both individuals diagnosed with breast cancer and in genomes of individuals diagnosed with prostate cancer, but not in genomes of individuals listed in Table 2, column “Cont-WGS,”
(d) pL1s identified in Table 2 as found only in individuals diagnosed with Alzheimer's Disease,
(e) pL1s identified in Table 2 as found by WGS, SCORE, or both, in individuals diagnosed with Alzheimer's Disease, in individuals diagnosed with breast cancer, and in individuals diagnosed with prostate cancer, but not in genomes of individuals listed in Table 2, column “Cont-WGS,” wherein, if said individual has one or more pL1s identified in groups (a)-(e), said individual is at risk of developing cancer or Alzheimer's Disease.

23. The method of claim 22, wherein said pL1s are of group (a), and the individual's risk is of breast cancer.

24. The method of claim 22, wherein said pL1s are of group (b), and the individual's risk is of prostate cancer.

25. The method of claim 22, wherein said pL1s are of group (c), and the individual's risk is of cancer in general, (if female) breast cancer in particular, or, (if male) prostate cancer in particular.

26. The method of claim 22, wherein said pL1s are of group (d), and the individual's risk is of Alzheimer's Disease.

27. The method of claim 22, wherein said pL1s are of group (e), and the individual's risk is of cancer or Alzheimer's Disease.

28. A method for determining how many polymorphic LINE-1 elements (“pL1s”) which pL1s have a 5′ untranslated region (“5′UTR”) and a 3′UTR, which 5′UTR begins with a contiguous sequence of at least 300 bases and which 3′UTR terminates in a contiguous sequence of at least 300 bases, may be full-length pL1s in genomic DNA of a subject who has both (a) pL1s, and (b) LINE-1 elements that occur at known genomic locations in all individuals (“fixed L1s”) with known genomic sequences upstream and downstream of said known genomic locations, with regard to pL1 insertions sites at which pL1s are shown in Table 2 to be:
(group 1) found to be inserted at said sites only in persons diagnosed with breast cancer,
(group 2) found to be inserted at said sites only in persons diagnosed with prostate cancer,
(group 3) found to be inserted at said sites in both persons diagnosed with breast cancer and in persons diagnosed with prostate cancer,
(group 4) found to be inserted at said sites only in individuals diagnosed with Alzheimer's Disease, or,
(group 5) found to be inserted at said sites in individuals diagnosed with Alzheimer's Disease, in individuals diagnosed with breast cancer, and in individuals diagnosed with prostate cancer, but not in genomes of individuals listed in Table 2, column “Cont-WGS”, said method comprising the following steps, in the following order:
(a) obtaining genomic DNA from said subject, which genomic DNA is fragmented into lengths of choice, and
(b) contacting said fragmented genomic DNA with
(1) a plurality of first DNA probes, first RNA probes, or a mixture of both first DNA probes and first RNA probes, each of which said first DNA probes and first RNA probes (A) comprises a contiguous sequence of about 200 to about 1000 bases complementary to a consensus human genomic sequence surrounding and including one particular known pL1 insertion site, wherein said plurality of said first DNA probes, first RNA probes, or mixture of both first DNA probes and first RNA probes taken together comprises human genomic sequence surrounding and including each of said pL1 insertion points in at least one of said groups (1) to (5), and (ii) wherein each of said first DNA probes and said first RNA probes is (A) attached to an solid support or (B) are tagged with a tag which allows said probes to be specifically captured on a solid support when desired, and
(2) a plurality of second DNA probes, second RNA probes, or mixture of both second DNA probes and second RNA probes, wherein said second DNA probes and said second RNA are complementary to said beginning contiguous sequence of said 300 bases of said 5′UTR of said pL1, further wherein each of said second DNA probe and second RNA probe is (A) attached to a solid support or (B) are tagged to allow said probes to be specifically captured on a support when desired,
under conditions allowing said fragmented genomic DNA complementary to any of said first DNA probes, first RNA probes, or a mixture of both first DNA probes and first RNA probes or to said second DNA probes, second RNA probes, or a mixture of both second DNA probes and second RNA probes to hybridize to said probes, thereby creating a mixture of unhybridized fragmented genomic DNA, and fragmented genomic DNA that has hybridized to one of said probes,
(c) if probes have been used in step (b) that are tagged to allow said tagged probes to be specifically captured on a solid support when desired, capturing said tagged probes on said solid support, or, if said probes were already attached to a solid support, proceeding to step (d),
(d) eluting any fragmented genomic DNA that has not hybridized to either one of said first DNA probes, first RNA probes, or mixture of both first DNA probes and first RNA probes, or one of said second DNA probes, second RNA probes, or mixture of both second DNA probes and second RNA probes,
(e) eluting from said supports and collecting for sequencing any fragmented genomic DNA that hybridized to one of said first DNA probes, first RNA probes, or a mixture of both first DNA probes and first RNA probes, or to said second DNA probes, second RNA probes, or mixture of both second DNA probes and second RNA probes, thereby obtaining a plurality of previously-hybridized genomic DNA fragments,
(f) sequencing said plurality of previously-hybridized genomic DNA fragments, thereby obtaining a DNA sequence for each fragment contained within said plurality of previously-hybridized genomic DNA fragments,
(g) comparing said DNA sequence for each fragment contained within plurality of previously-hybridized genomic DNA fragments to consensus human genomic sequences including each of said pL1 insertion sites for said in at least one of said groups (1) to (5), and determining for each of said pL1 insertion sites in said at least one of said groups (1) to (5) whether:
(1) said genomic sequence upstream for each of said pL1 insertion sites is followed by (i) some or all of beginning of said L1 5′UTR sequence or (ii) some or all of said end of said L1 3′ sequence,
indicating that for those insertion sites, there is a pL1 present that may be full length, and
(2) whether said genomic sequence downstream for each of said pL1 insertion sites set forth in Table 2 is followed by (i) some or all of beginning of said L1 5′UTR sequence or (ii) some or all of said end of said L1 3′ UTR sequence,
indicating that for those pL1 insertion sites, there is a pL1 present that may be full length.

29. The method of claim 28, further comprising step (g)(3), compiling a list of how many pL1s that have said beginning of said L1 5′UTR and said end of said L1 3′UTR are present in said genome from said individual, thereby determining how many pL1s in said at least one of said groups (1) to (5) may be full-length.

30. The method of claim 29, further comprising step (g)(4), identifying in said list the locations of each of said pL1s in said at least one of said groups (1) to (5) present in said individual.

31. The method of claim 30, further comprising step (g)(5), for each location in which a pL1 has been identified in step (g)(4), determining whether (A) said plurality of sequenced DNA sequences also contains a normal genomic sequence uninterrupted by a pL1 at said location, thereby determining that there is a copy of pL1 and a normal genomic sequence at that location, indicating that said genome of said individual has one copy of genomic sequence with said pL1 at said genomic location and one copy that does not have a pL1 at said location, or (B) said plurality of sequenced DNA sequences do not also contain a normal genomic sequence uninterrupted by a pL1 at said location, indicating that the genome of said individual has two copies of genomic sequence with said pL1 at said genomic location.

32. The method of claim 31, further comprising steps:
(h)(1), comparing the genomic sequences upstream and downstream of all L1 sequences in said plurality of sequenced DNA sequences to the genomic sequence upstream and downstream of said fixed L1s in said individual,
(h)(2), determining how many fixed L1s have been detected compared to the number known to exist in the human genome, and
(h)(3) reporting whether the number of fixed L1s detected in said individual is the same or different from the number of fixed L1s known to exist in said human genome.

33-37. (canceled)

38. A kit for determining with regard to a human genome having a genomic sequence proceeding in direction from 5′ to 3′, which genome has 826 known potential insertion points at which a full-length polymorphic LINE-1 element (“pL1”) may be inserted which of said insertion point has had a pL1 inserted, said full-length pL1s having a 5′ untranslated region (“5′UTR”) and a 3′UTR, which 5′UTR begins with a contiguous sequence of at least 300 bases and which 3′UTR terminates in a contiguous sequence, said kit comprising
(a) a set of probes for a subset of said 826 potential insertion points listed in Table 2, said subset consisting of one or more of said following groups:
group 1: pL1 insertions sites at which pL1s are shown in Table 2 to be found to be inserted at said sites only in persons diagnosed with breast cancer,
group 2: pL1 insertions sites at which pL1s are shown in Table 2 found to be inserted at said sites only in persons diagnosed with prostate cancer,
group 3: pL1 insertions sites at which pL1s are shown in Table 2 found to be inserted at said sites in both persons diagnosed with breast cancer and in persons diagnosed with prostate cancer,
group 4: pL1 insertions sites at which pL1s are shown in Table 2 found to be inserted at said sites only in individuals diagnosed with Alzheimer's Disease, and,
group 5: pL1 insertions sites at which pL1s are shown in Table 2 found to be inserted at said sites in individuals diagnosed with Alzheimer's Disease, in individuals diagnosed with breast cancer, and in individuals diagnosed with prostate cancer, but not in genomes of individuals listed in Table 2, column “Cont-WGS”,
each member of which set of probes comprises (i) a sequence complementary to genomic sequence contiguous to one of said insertion points at which pL1 inserts into said genome, attached directly to a sequence complementary to at least the first 100 bases of said beginning of said 5′UTR of said pL1.

39. The kit of claim 38, further comprising (b) probes consisting essentially of 100-600 contiguous bases of said pL1 5′UTR.

40-42. (canceled)
